(19) United States
(12) Patent Application Publication
(10) Pub. No.: US 2020/0377945 A1
(43) Pub. Date: Dec. 3, 2020

(54) BIOMARKERS FOR TYPE 2 DIABETES MELLITUS AND USE THEREOF

(71) Applicant: PERFECT (CHINA) CO., LTD, Guangdong (CN)

(72) Inventors: Liping ZHAO, Shanghai (CN); Chenhong ZHANG, Shanghai (CN); Guojun WU, Shanghai (CN); Menghui ZHANG, Shanghai (CN)

(73) Assignee: PERFECT (CHINA) CO., LTD, Guangdong (CN)

(21) Appl. No.: 16/261,037

(22) Filed: Jan. 29, 2019

(30) Foreign Application Priority Data
Jan. 31, 2018 (CN) 201810143729.2

(51) Int. Cl. C12Q 1/6883

(52) U.S. Cl. CPC C12Q 1/6883; C12Q 2600/158; C12Q 2600/106

(57) ABSTRACT

The present invention provides a method for assessing the presence or the risk of development of type 2 diabetes mellitus in a subject based on abundance data of several CAGs. Also provided is a method for evaluating efficacy of diet intervention or disease treatment in a subject having type 2 diabetes mellitus based on abundance data of these CAGs.

The present application contains a Sequence Listing that has been submitted electronically in ASCII format and is hereby incorporated by reference in its entirety. The ASCII copy, created on Jan. 17, 2020, is named 184627 Substitute Sequence Listing_ST25.txt and is 252,667,892 bytes in size.

### BACKGROUND

The gut microbiota provides many beneficial functions to the human host. Some of these functions are essential to us as we do not encode them in our own genome. From an ecological perspective, such functions can be considered as “ecosystem services” (1). Function-wise, a “healthy” gut microbiota is one that is able to provide all the ecosystem services that are required. Short-chain fatty acid (SCFA) production is the most notable example of such service provided by the gut bacteria. There is already a large body of literature on how humans may directly benefit from SCFAs, e.g. butyrate is the primary energy substrate for colonocytes and a wide range of SCFAs function as signaling molecules that modulate inflammation and appetite regulation (2). Bacteria that supply SCFAs to humans are therefore the ecosystem service providers (ESPs) and the key members of the gut microbiota for keeping the human host healthy.

Deficiency of SCFA producers has been linked to dysbiosis-related diseases such as type 2 diabetes mellitus (T2DM) (3-6). Clinical trials using high dietary fibre diets have been shown to alleviate the disease phenotypes of T2DM but with vastly different treatment response across individuals (7-9), potentially due to person-specific profiles of SCFA producers in the gut microbiota (10).

Identifying ESPs for SCFA production to ameliorate T2DM, however, is no easy task. The capacity for fermenting organic compounds into SCFAs is a genetic trait shared by hundreds of gut bacterial species across many taxa (11). Some SCFA producers may outcompete others due to different tolerance to acidity in the gut lumen (12, 13). This presents the need to make a distinction between a “producer”, which has the genetic capacity for producing SCFAs, and a “provider”, which indeed ferments carbohydrates and supplies SCFAs in the specific gut environment. Our recent studies further demonstrated a strain-specific response in butyrate- and acetate-producing species to a high dietary fibre diet (14, 15). This calls for a strain-level microbiome-wide association approach to identify the ESPs which are the actual suppliers of SCFAs to the human host in response to high dietary fibre intake.

### SUMMARY OF THE INVENTION

The present application uses shotgun metagenomic sequencing to reveal the changes of gut microbiome in T2D patients in response to high-fibre intervention. As a result, 15 CAGs (co-abundance gene groups), and designated as CAG NO.: 1 to 15, were found to be upregulated and identified as ESPs, while 49, designated as CAG NO.: 16 to 64, were downregulated in T2D patients. These CAGs can be used as the biomarkers for efficient, accurate and patient friendly characterization of T2D.

In one aspect, the present invention provides a method for assessing the presence or the risk of development of type 2 diabetes mellitus in a subject, comprising the steps of:
a) collecting a fecal sample from the subject;
b) analyzing DNA extracted from the fecal sample to determine abundance of each reference CAG selected from the group consisting of CAG Nos.: 1-64,

Aᵢ (abundance of CAG No: i)=number of reads aligned to CAG No.: i/(size of CAG No.: i×number of total reads);
c) calculating GMM-index of each sample using the calculated abundance data,

GMM-index=log (ρᵢ₌₁¹⁵Aᵢ/Σᵢ₌₁₆⁶⁴Aᵢ); and
d) determining that the subject suffers from or at a risk of developing type 2 diabetes mellitus if the GMM-index is close to or lower than a predetermined level, wherein, CAG NOs.:1-15 comprise nucleic acid sequences set forth in SEQ ID NOs.: 1-191, 192-326, 327-593, 594-835, 836-885, 886-960, 961-1097, 1098-1264, 1265-1433, 1434-1684, 1685-1833, 1834-1979, 1980-2163, 2164-2447, and 2448-2783, respectively, and CAG NOs.:16-64 comprise nucleic acid sequences set forth in SEQ ID NOs.: 2784-2961, 2962-3130, 3131-3525, 3526-3747, 3748-3863, 3864-4068, 4069-4212, 4213-4393, 4394-4532, 4533-4891, 4892-4979, 4980-5116, 5117-5320, 5321-5464, 5465-5781, 5782-6279, 6280-6646, 6647-6954, 6955-7178, 7179-7613, 7614-7758, 7759-8046, 8047-8491, 8492-8546, 8547-9971, 9972-10099, 10100-10392, 10393-10502, 10503-10694, 10695-10986, 10987-11089, 11090-11262, 11263-11466, 11467-11704, 11705-12034, 12035-12113, 12114-12341, 12342-12454, 12455-12664, 12665-12825, 12826-13042, 13403-13500, 13501-13726, 13727-13949, 13950-14014, 14015-14290, 14291-14403, 14404-14686, and 14687-14850, respectively.

In some embodiments, analysis of DNA in step b) comprises the steps of obtaining the DNA sequences and aligning the obtained DNA sequences with the nucleic acid sequences set forth in SEQ ID Nos.: 1-14850.

In some embodiments, obtaining of DNA sequences comprises the steps of obtaining raw sequence reads in the sample and processing the raw sequence reads to obtain qualified sequence reads.

In some embodiments, the raw sequence reads are obtained by a PCR-based high-throughput sequencing technique. In some embodiments, the raw sequence reads are obtained by Illumina sequencing.

In some embodiments, the processing of the raw sequence reads comprises removal of adapters, trimming of sequences at 3′ end until reaching the first nucleotide with a quality threshold higher than 20, removal of short sequences, and removal of sequences aligned to human genome. In some embodiments, the short sequences are 59 bp or less in length.

In some embodiments, the alignment of DNA sequences uses seed-and-extend strategy. In some embodiments, the sequences with no mismatch in seed sequence are used to determine the abundance of each reference CAG in step b). In some embodiments, the length of the seed sequence is 4 bp or more, 5 bp or more, 6 bp or more, 7 bp or more, 8 bp or more, 9 bp or more, 10 bp or more, 11 bp or more, 12 bp or more, 13 bp or more, 14 bp or more, 15 bp or more, 16 bp or more, 17 bp or more, 18 bp or more, or 19 bp or more. In some embodiments, the length of the seed sequence is 31 bp or less, 30 bp or less, 29 bp or less, 28 bp or less, 27 bp or less, 26 bp or less, 25 bp or less, 24 bp or less, 23 bp or less, 22 bp or less, or 21 bp or less. In some embodiments, the seed sequence is 20 bp in length.

In some embodiments, the predetermined level is approximately −1.028883.

In a second aspect, the instant invention provides a method for evaluating efficacy of diet intervention or disease treatment in a subject having type 2 diabetes mellitus, comprising the steps of
a) collecting a fecal sample from the subject before and during the diet intervention or disease treatment;
b) analyzing DNA extracted from the fecal sample to determine abundance of each reference CAG selected from the group consisting of CAG Nos.: 1-64,

Aᵢ (abundance of CAG No: i)=number of reads aligned to CAG No.: i/(size of CAG No.: i×number of total reads);
c) calculating GMM-index of each sample using the calculated abundance data,

GMM-index=log (ρᵢ₌₁¹⁵Aᵢ/Σᵢ₌₁₆⁶⁴ Aᵢ); and
e) determining that the subject responds positively to the diet intervention or disease treatment if the GMM-index is increased in the sample collected during the diet intervention or disease treatment,
   wherein, CAG NOs.:1-15 comprise nucleic acid sequences set forth in SEQ ID NOs.: 1-191, 192-326, 327-593, 594-835, 836-885, 886-960, 961-1097, 1098-1264, 1265-1433, 1434-1684, 1685-1833, 1834-1979, 1980-2163, 2164-2447, and 2448-2783, respectively, and CAG NOs.:16-64 comprise nucleic acid sequences set forth in SEQ ID NOs.: 2784-2961, 2962-3130, 3131-3525, 3526-3747, 3748-3863, 3864-4068, 4069-4212, 4213-4393, 4394-4532, 4533-4891, 4892-4979, 4980-5116, 5117-5320, 5321-5464, 5465-5781, 5782-6279, 6280-6646, 6647-6954, 6955-7178, 7179-7613, 7614-7758, 7759-8046, 8047-8491, 8492-8546, 8547-9971, 9972-10099, 10100-10392, 10393-10502, 10503-10694, 10695-10986, 10987-11089, 11090-11262, 11263-11466, 11467-11704, 11705-12034, 12035-12113, 12114-12341, 12342-12454, 12455-12664, 12665-12825, 12826-13042, 13403-13500, 13501-13726, 13727-13949, 13950-14014, 14015-14290, 14291-14403, 14404-14686, and 14687-14850, respectively.

In some embodiments, analysis of DNA in step b) comprises the steps of obtaining the DNA sequences and aligning the obtained DNA sequences with the nucleic acid sequences set forth in SEQ ID Nos.: 1-14850.

In some embodiments, obtaining of DNA sequences comprises the steps of obtaining raw sequence reads in the sample and processing the raw sequence reads to obtain qualified sequence reads.

In some embodiments, the raw sequence reads are obtained by a PCR-based high-throughput sequencing technique. In some embodiments, the raw sequence reads are obtained by Illumina sequencing.

In some embodiments, the processing of the raw sequence reads comprises removal of adapters, trimming of sequences at 3′ end until reaching the first nucleotide with a quality threshold higher than 20, removal of short sequences, and removal of sequences aligned to human genome. In some embodiments, the short sequences are 59 bp or less in length.

In some embodiments, the alignment of DNA sequences uses seed-and-extend strategy. In some embodiments, the sequences with no mismatch in seed sequence are used to determine the abundance of each reference CAG in step b). In some embodiments, the length of the seed sequence is 4 bp or more, 5 bp or more, 6 bp or more, 7 bp or more, 8 bp or more, 9 bp or more, 10 bp or more, 11 bp or more, 12 bp or more, 13 bp or more, 14 bp or more, 15 bp or more, 16 bp or more, 17 bp or more, 18 bp or more, or 19 bp or more. In some embodiments, the length of the seed sequence is 31 bp or less, 30 bp or less, 29 bp or less, 28 bp or less, 27 bp or less, 26 bp or less, 25 bp or less, 24 bp or less, 23 bp or less, 22 bp or less, or 21 bp or less. In some embodiments, the seed sequence is 20 bp in length.

In one embodiment, during the diet intervention or disease treatment, the fecal sample is collected one week, two weeks, three weeks, and/or four weeks after the diet intervention or disease treatment begins.

In some embodiments, the subject is determined to respond positively to the diet intervention or disease treatment when the GMM-index becomes close to or higher than a predetermined level during the diet intervention or disease treatment. In some embodiments, the predetermined level is −1.028883.

In a third aspect, the present invention provides a method for assessing the presence or the risk of development of type 2 diabetes mellitus in a subject, comprising the steps of:
a) collecting a fecal sample from the subject;
b) analyzing DNA extracted from the fecal sample to determine abundance of each reference CAG selected from the group consisting of CAG ID Nos.: 1-15,

Aᵢ (abundance of CAG No.: i)=number of reads aligned to the CAG No.: i/(size of CAG No.: i×number of total reads);
c) calculating ESP-index of each sample using the calculated abundance data,

ESP-Index=In(Help×10¹⁰×Σᵢ₌₁¹⁵Aᵢ), wherein Heip=(e^{H}−1)/14, H=−ρᵢ₌₁¹⁵ AᵢInAᵢ; and
d) determining that the subject suffers from or at a risk of developing type 2 diabetes mellitus if the ESP-index is close to or lower than a predetermined level,
   wherein, CAG NOs.:1-15 comprise nucleic acid sequences set forth in SEQ ID NOs.: 1-191, 192-326, 327-593, 594-835, 836-885, 886-960, 961-1097, 1098-1264, 1265-1433, 1434-1684, 1685-1833, 1834-1979, 1980-2163, 2164-2447, and 2448-2783, respectively.

In some embodiments, analysis of DNA in step b) comprises the steps of obtaining the DNA sequences and aligning the obtained DNA sequences with the nucleic acid sequences set forth in SEQ ID Nos.: 1-2783.

In some embodiments, obtaining of DNA sequences comprises the steps of obtaining raw sequence reads in the sample and processing the raw sequence reads to obtain qualified sequence reads.

In some embodiments, the raw sequence reads are obtained by a PCR-based high-throughput sequencing technique. In some embodiments, the raw sequence reads are obtained by Illumina sequencing.

In some embodiments, the processing of the raw sequence reads comprises removal of adapters, trimming of sequences at 3′end until reaching the first nucleotide with a quality threshold higher than 20, removal of short sequences, and removal of sequences aligned to human genome. In some embodiments, the short sequences are 59 bp or less in length.

In some embodiments, the alignment of DNA sequences uses seed-and-extend strategy. In some embodiments, the sequences with no mismatch in seed sequence are used to determine the abundance of each reference CAG in step b). In some embodiments, the length of the seed sequence is 4 bp or more, 5 bp or more, 6 bp or more, 7 bp or more, 8 bp or more, 9 bp or more, 10 bp or more, 11 bp or more, 12 bp or more, 13 bp or more, 14 bp or more, 15 bp or more, 16 bp or more, 17 bp or more, 18 bp or more, or 19 bp or more. In some embodiments, the length of the seed sequence is 31 bp or less, 30 bp or less, 29 bp or less, 28 bp or less, 27 bp or less, 26 bp or less, 25 bp or less, 24 bp or less, 23 bp or less, 22 bp or less, or 21 bp or less. In some embodiments, the seed sequence is 20 bp in length.

In some embodiments, the predetermined level is approximately 4.4.

In a fourth aspect, the instant invention provides a method for evaluating efficacy of diet intervention or disease treatment in a subject having type 2 diabetes mellitus, comprising the steps of
a) collecting a fecal sample from the subject before and during the diet intervention or disease treatment;
b) analyzing DNA extracted from the fecal sample to determine abundance of each reference CAG selected from the group consisting of CAG ID Nos.: 1-15,

Aᵢ (abundance of CAG No.: i)=number of reads aligned to CAG No.: i/(size of CAG No.: i×number of total reads);
c) calculating ESP-index of each sample using the calculated abundance data,

ESP-Index=In(Heip×10¹⁰×Σᵢ₌₁¹⁵Aᵢ), wherein Heip=(e^{H}−1)/14, H=−Σᵢ₌₁¹⁵AᵢInAᵢ; and
e) determining that the subject responds positively to the diet intervention or disease treatment if the ESP-index is increased in the sample collected during the diet intervention or disease treatment,
   wherein, CAG NOs.:1-15 comprise nucleic acid sequences set forth in SEQ ID NOs.: 1-191, 192-326, 327-593, 594-835, 836-885, 886-960, 961-1097, 1098-1264, 1265-1433, 1434-1684, 1685-1833, 1834-1979, 1980-2163, 2164-2447, and 2448-2783, respectively.

In some embodiments, analysis of DNA in step b) comprises the steps of obtaining the DNA sequences and aligning the obtained DNA sequences with the nucleic acid sequences set forth in SEQ ID Nos.: 1-2783.

In some embodiments, obtaining of DNA sequences comprises the steps of obtaining raw sequence reads in the sample and processing the raw sequence reads to obtain qualified sequence reads.

In some embodiments, the raw sequence reads are obtained by a PCR-based high-throughput sequencing technique. In some embodiments, the raw sequence reads are obtained by Illumina sequencing.

In some embodiments, the processing of the raw sequence reads comprises removal of adapters, trimming of sequences at 3′end until reaching the first nucleotide with a quality threshold higher than 20, removal of short sequences, and removal of sequences aligned to human genome. In some embodiments, the short sequences are 59 bp or less in length.

In some embodiments, the alignment of DNA sequences uses seed-and-extend strategy. In some embodiments, the sequences with no mismatch in seed sequence are used to determine the abundaned of each reference CAG in step b). In some embodiments, the length of the seed sequence is 4 bp or more, 5 bp or more, 6 bp or more, 7 bp or more, 8 bp or more, 9 bp or more, 10 bp or more, 11 bp or more, 12 bp or more, 13 bp or more, 14 bp or more, 15 bp or more, 16 bp or more, 17 bp or more, 18 bp or more, or 19 bp or more. In some embodiments, the length of the seed sequence is 31 bp or less, 30 bp or less, 29 bp or less, 28 bp or less, 27 bp or less, 26 bp or less, 25 bp or less, 24 bp or less, 23 bp or less, 22 bp or less, or 21 bp or less. In some embodiments, the seed sequence is 20 bp in length.

In one embodiment, during the diet intervention or disease treatment, the fecal sample is collected one week, two weeks, three weeks, and/or four weeks after the diet intervention or disease treatment begins.

In some embodiments, the subject is determined to respond positively to the diet intervention or disease treatment when the ESP-index becomes close to or higher than a predetermined level during the diet intervention or disease treatment. In some embodiments, the predetermined level is 4.4.

In a fifth aspect, the instant application provides a microbe, comprising one or more of a bacteria corresponding-CAG NO.1-15, wherein CAG NO.1-15 comprises nucleic acids set forth in SEQ ID NO.: 1-191, 192-326, 327-593, 594-835, 836-885, 886-960, 961-1097, 1098-1264, 1265-1433, 1434-1684, 1685-1833, 1834-1979, 1980-2163, 2164-2447, and 2448-2783 respectively.

Other features and advantages of the instant disclosure will be apparent from the following detailed description and examples, which should not be construed as limiting. The contents of all references, Genbank entries, patents and published patent applications cited throughout this application are expressly incorporated herein by reference.

### BRIEF DESCRIPTION OF THE DRAWINGS

FIG. 1 shows the profile of the clinical trial in the Example.

FIGS. 2A, 2B, and 2C show that a high dietary fibre diet alters the gut microbiota and improves glucose homeostasis in patients with type 2 diabetes. (A) Changes in the circulating parameters of glucose homeostasis (HbAlc, fasting blood glucose, glucose and insulin area-under-curve (AUC) in meal tolerance test (MTT)). Data were presented as percentage changes from Day 0 (±standard errors). Two-way repeated measures analysis of variance with a Tukey's post-hoc test was used for intra- and inter-group comparisons. *P <0.05, **P <0.01 and ***P <0.001 vs Day 0 of the same group; #P <0.05, ##P <0.01 and ###P <0.001 vs U group at the same time point. N=27 for W and n=16 for U group for all analyses except n=15 in the U group for MTT. (B) Changes in the overall gut microbial structure. Principal coordinate analysis was performed based on the Bray-Curtis distance for 422 bacterial co-abundance gene groups. (C) Changes in the gut microbiota diversity (gene richness). The change in gene counts was adjusted to 31 million mapped reads per sample. Data were shown as the mean±S.E.M. Boxes showed the medians and the interquartile ranges, the whiskers denoted the lowest and highest values that are within 1.5 times the IQR from the first and third quartiles, and outliers were shown as individual points. Wilcoxon matched-pair signed-rank tests (two-tailed) were used to analyse each pair-wise comparison within each group. Mann-Whitney test was used to analyse differences between the W and U groups at the same time point. *P <0.05, **P <0.01 and ***P <0.001 (Adjusted by Benjamini & Hochberg, 1995). W=acarbose plus WTP diet; U=acarbose plus usual care (control).

FIGS. 3A, 3B, 3C, and 3D show that transplantation of dietary fibre-supplemented gut microbiota improves glucose tolerance in mice. (A) Body weight, (B) fasting blood glucose (FBG), (C) oral glucose tolerance test (2 weeks after transplantation) and (D) fasting circulating insulin of germ-free mice receiving faecal microbiota transplantation. The transplant material was derived from representative donors, one from W and one from U group, both before (“Pre”; Day 0) and after (“Post”; Day 84) the intervention. Mice receiving transplant: n=5 for W-Pre, W-Post, U-Pre and n=4 for U-Post. *P <0.05, **P <0.01 and ***P <0.001 using one-way ANOVA with a Tukey's post-hoc test for intra- and inter-group comparisons. W=acarbose plus WTP diet; U=acarbose plus usual care (control).

FIGS. 4A and 4B show heat maps indicating the abundance (log-transformed) of intervention-responsive bacteria within the (A) W or (B) U group (Wilcoxon matched-pair signed-rank tests were used to compare the data on Day 0 and Day 28. P <0.05, Adjusted by Benjamini & Hochberg, 1995). The bacteria were clustered with a Spearman correlation coefficient and ward linkage. For W, n=27; for U, n=16.

FIGS. 5A, 5B, 5C, 5D, and 5E show potential ecosystem service providers (ESPs) and the co-excluded detrimental bacteria. The distribution networks of genes involved in production of short-chain fatty acids (SCFAs), H₂S and indole in 154 high quality draft genomes are shown for genomes that (A) decreased or (B) increased in abundance following intervention in the W group, or that (C) decreased or (D) increased in abundance following intervention in the U group. The histograms next to each grey circle (high quality draft genome identified as a bacterial strain) represent the mean abundance (log-transformed) at Day 0 and Day 28. Changes in bacterial abundance were determined according to those in FIG. 4. Lines connecting the grey circles to other shapes indicate genes involved in specific activities. Brown triangles indicate genes involved in H₂5 production; purple parallelograms indicate genes involved in indole production; green and blue shapes indicate genes involved in SCFA production. Acetic acid synthesis: formate-tetrahydrofolate ligase. Butyric acid synthesis: butyryl-CoA:acetate CoA transferase (But); butyryl-CoA:acetoacetate CoA transferase (Ato; consisting of alpha (AtoA) and beta (AtoD) subunits); butyrate kinase (Buk); butyryl-CoA: 4-hydroxybutyrate CoA transferase (4Hbt). Propanoic acid synthesis: propionateCoA-transferase/propionyl-CoA: succinate-CoA transferase (PCoAt). (E) Changes of the abundance of ecosystem service providers. The size and colour of the circles indicated the average abundance and coefficient of variance of the abundance of the strain respectively. W=acarbose plus WTP diet; U=acarbose plus usual care (control).

FIGS. 6A, 6B, and 6C show that high fibre diet reduces endotoxin load and inflammation. (A) Lipopolysaccharide binding protein. (B) White blood cell count. (C) TNF-a. A two-way repeated measures analysis of variance with the Tukey post-hoc test was used for intra- and inter-group comparisons. * P <0.05, ** P <0.01, *** P <0.001 vs Day 0 of the same group; #P <0.05, ##P <0.01, ###P <0.001 vs U group at the same time point. N=27 for W and n=16 for U group. W=acarbose plus WTP diet; U=acarbose plus usual care (control).

FIGS. 7A, 7B, 7C, and 7D show correlation between abundance of the bacterial CAGs and alleviation of phenotypes of type 2 diabetes mellitus. (A-B) Heat maps calculated from Spearman correlation coefficients between abundance of bacterial CAGs and levels of clinical variables in the W (A) and U groups (B) *=P <0.05, **=P <0.01 (Adjusted by Benjamini & Hochberg, 1995). The bacteria were clustered with a Spearman correlation coefficient and ward linkage based on their amounts. (C) In GUT2DM project, the post-intervention level of HbAlc was negatively correlated (Spearman correlation coefficient (SCC)=−0.4901, P=1.0253e⁻¹¹) with the Gut Microbiota Modulation (GMM) index of the 15 EPSs that increased divided by the abundance of the 49 that decreased in the training dataset (27 patients in the W group and 16 in the U group). (D) In the testing QIDONG clinical trial, the post-intervention level of HbAlc was also negatively correlated (SCC=−0.4006, P=4.53e⁻⁷) with the Gut Microbiota Modulation (GMM) index of the 15 EPSs and their 49 co-excluding bacteria in a testing data set of 74 patients who all received a high-fibre diet without acarbose for 3 months.

FIGS. 8A, 8B, 8C, 8D, and 8E show that abundance and diversity of the ecosystem service providers (ESPs) correlate with alleviation of disease phenotypes in patients with type 2 diabetes. (A) Heat maps for correlation between abundance of individual ESP and clinical variables. *P <0.05 and **P <0.01. (B) Changes in the ESP-Index (ln(Heip×10¹⁰×Σ_{i32 1}¹⁵ Aᵢ), where Aᵢ is the abundance of ESPᵢ). (C) Correlation between the ESP-index (Day 0 and Day 84) and HbAlc (Day 0 and Day 84) in the GUT2D study. N=43. (D) Correlation between the ESP-index (Day 0 and Day 28) and HbAlc (Day 0 and Day 84) in the GUT2D study. N=43. (E) Correlation between the ESP-index (Day 0 and Day 84) and HbAlc (Day 0 and Day 84) in the QIDONG study. N=71. All correlation coefficients were calculated using the method described by Bland and Altman (16). W=acarbose plus WTP diet; U=acarbose plus usual care (control).

### DETAILED DESCRIPTION OF THE INVENTION

In order that the present disclosure may be more readily understood, certain terms are defined here. Additional definitions are set forth throughout the detailed description.

The term “co-abundance gene group” or “CAG” refers to groups of genes that correlate in terms of abundance to randomly picked seed genes. Segregating a metagenome into groups of genes that have similar abundance allows the identification of biological entities like prokaryotes and phages, as well as small genetic entities representing co-inherited clonal heterogeneity.

The term “size of CAG No.: i” used herein refers to the length of CAG No.: i, i.e., the number of nucleotides of CAG No.: i.

The term “biomarker” refers to a measurable indicator of some biological state or condition. The biomarker used herein is the CAG, the abundance data of which may be indicative of T2D.

The term “Receiver operating characteristic curve” or “ROC curve” used herein refers to a graphical plot that illustrates the diagnostic ability of a binary classifier system as its discrimination threshold is varied. The ROC curve is created by plotting the true positive rate against the false positive rate at various threshold settings. The true-positive rate is also known as sensitivity, recall or probability of detection. The false-positive rate is also known as the fall-out or probability of false alarm and can be calculated as (1-specificity). The ROC curve is thus the sensitivity as a function of fall-out.

The term “Youden's index” refers to the difference between the true positive rate and the false positive rate. Maximizing this index allows to find, from the ROC curve, an optimal cut-off point independently from the prevalence. The index is represented graphically as the height above the chance line.

The term “area under the ROC curve” or “AUC” used herein is used to indicate the accuracy of a test which separates a group being tested into those with and without the disease in question.

In the present invention, with the scanning of whole gut microbiome, several CAGs have been found to be prevalently distributed in samples from the T2D patients that are responsive to high fibre diet intervention. Among these CAGs, 15 are upregulated while 49 are downregulated. The GMM-index and the ESP-index calculated based on the abundances of these or some of these CAGs in a fecal sample may be used to assess the presence or the risk of development of T2D in a subject. Alternatively, the abundance changes of these or some of these CAGs may be used to monitor response to disease treatment or diet intervention in a patient having T2D. Both methods can be performed in an efficient, accurate and patient friendly manner.

The present invention provides a method for assessing the presence or the risk of development of type 2 diabetes mellitus in a subject, comprising the steps of:
a) collecting a fecal sample from the subject;
b) analyzing DNA extracted from the fecal sample to determine abundance of each reference CAG selected from the group consisting of CAG ID Nos.: 1-64,

Aᵢ (abundance of CAG No: i)=number of reads aligned to CAG No.: i/(size of CAG No.: i×number of total reads);
c) calculating GMM-index of each sample using the calculated abundance data,

GMM-index=log (Σᵢ₌₁¹⁴Aᵢ/Σᵢ₌₁₆⁶⁴Aᵢ); and
d) determining that the subject suffers from or at a risk of developing type 2 diabetes mellitus if the GMM-index is close to or lower than a predetermined level.

The instant invention provides a method for evaluating efficacy of diet intervention or disease treatment in a subject having type 2 diabetes mellitus, comprising the steps of
a) collecting a fecal sample from the subject before and during the diet intervention or disease treatment;
b) analyzing DNA extracted from the fecal sample to determine abundance of each reference CAG selected from the group consisting of CAG ID Nos.: 1-64,

Aᵢ (abundance of CAG No: i)=number of reads aligned to CAG No.: i/(size of CAG No.: i×number of total reads);
c) calculating GMM-index of each sample using the calculated abundance data,

GMM-index=log (Σᵢ₌₁¹⁵Aᵢ/Σᵢ₌₁₆⁶⁴Aᵢ); and
e) determining that the subject responds positively to the diet intervention or disease treatment if the GMM-index is increased in the sample collected during the diet intervention or disease treatment.

For the ESP-index aspect, the the present invention provides a method for assessing the presence or the risk of development of type 2 diabetes mellitus in a subject, comprising the steps of:
a) collecting a fecal sample from the subject;
b) analyzing DNA extracted from the fecal sample to determine abundance of each reference CAG selected from the group consisting of CAG ID Nos.: 1-15,

Aᵢ (abundance of CAG No.: i)=number of reads aligned to CAG No.: i/(size of CAG No.: i×number of total reads);
c) calculating ESP-index of each sample using the calculated abundance data,

ESP-Index=ln(Heip×10¹⁰×Σᵢ₌₁¹⁵Aᵢ), wherein Heip=(e^{H}−1)/14, H=−Σᵢ₌₁¹⁵AᵢlnAᵢ and
d) determining that the subject suffers from or at a risk of developing type 2 diabetes mellitus if the ESP-index is close to or lower than a predetermined level,

The instant invention further provides a method for evaluating efficacy of diet intervention or disease treatment in a subject having type 2 diabetes mellitus, comprising the steps of
a) collecting a fecal sample from the subject before and during the diet intervention or disease treatment;
b) analyzing DNA extracted from the fecal sample to determine abundance of each reference CAG selected from the group consisting of CAG ID Nos.: 1-15,

Aᵢ (abundance of CAG No.: i)=number of reads aligned to CAG No.: i/(size of CAG No.: i×number of total reads);
c) calculating ESP-index of each sample using the calculated abundance data,

ESP-Index=ln(Heip×10¹⁰×Σᵢ₌₁¹⁵Aᵢ), wherein Heip=(e^{H}−1)/14, H=−Σᵢ₌₁¹⁵AᵢlnAᵢ and
e) determining that the subject responds positively to the diet intervention or disease treatment if the ESP-index is increased in the sample collected during the diet intervention or disease treatment.

In the present invention, CAG NOs.:1-15 comprise nucleic acid sequences set forth in SEQ ID NOs.: 1-191, 192-326, 327-593, 594-835, 836-885, 886-960, 961-1097, 1098-1264, 1265-1433, 1434-1684, 1685-1833, 1834-1979, 1980-2163, 2164-2447, and 2448-2783, respectively, and CAG NOs.:16-64 comprise nucleic acid sequences set forth in SEQ ID NOs.: 2784-2961, 2962-3130, 3131-3525, 3526-3747, 3748-3863, 3864-4068, 4069-4212, 4213-4393, 4394-4532, 4533-4891, 4892-4979, 4980-5116, 5117-5320, 5321-5464, 5465-5781, 5782-6279, 6280-6646, 6647-6954, 6955-7178, 7179-7613, 7614-7758, 7759-8046, 8047-8491, 8492-8546, 8547-9971, 9972-10099, 10100-10392, 10393-10502, 10503-10694, 10695-10986, 10987-11089, 11090-11262, 11263-11466, 11467-11704, 11705-12034, 12035-12113, 12114-12341, 12342-12454, 12455-12664, 12665-12825, 12826-13042, 13403-13500, 13501-13726, 13727-13949, 13950-14014, 14015-14290, 14291-14403, 14404-14686, and 14687-14850, respectively.

To determine abundance of each reference CAG of the present invention, any method well known in the art can be used. In some embodiments, DNA sequences are obtained from the fecal samples and then aligned with the CAG sequences. In some embodiments, seed-and-extend strategy is used in the alignment of DNA sequences, and the sequences with no mismatch in seed sequences are used to determine the abundance of each reference CAG. In some embodiments, the seed sequence is 20 bp in length.

The obtaining of DNA sequences comprises obtaining raw sequence reads in the sample and processing the raw sequence reads to obtain qualified sequence reads. In some embodiments, the raw sequence reads are obtained by a PCR-based high-throughput sequencing technique. In some embodiments, the raw sequence reads are obtained by Illumina sequencing. The processing of the raw sequence reads may be performed as known in the art. In some instances, the processing comprises removal of adapters, trimming of sequences at 3′end until reaching the first nucleotide with a quality threshold higher than 20, removal of short sequences, and removal of sequences aligned to human genome. In some embodiments, the short sequences are 59 bp or less in length.

In the method for assessing the presence or the risk of development of T2D in a subject, the subject is determined to suffer from or at a risk of developing T2D if the GMM-index or the ESP-index is close to or lower than a predetermined level.

The predetermined level can be set according to laboratory or clinical data. Even a level is predetermined, the hospital or the doctor may adjust it according to a subject's age, sex, physical conditions and the like.

In a preferred embodiment of the present invention, the predetermined level is approximately -1.028883 for the GMM-index. In a preferred embodiment of the present invention, the predetermined level is approximately 4.4 for the ESP-index. These specific levels are determined based on the Receiver operating characteristic curves, which have been created using data described hereinafter in the Examples. As described above, the Receiver operating characteristic curve is a graphical plot that illustrates the diagnostic ability of a binary classifier system as its discrimination threshold is varied. And Youden's index refers to the difference between the true positive rate and the false positive rate. Youden's index is often used in conjunction with Receiver Operating Characteristic (ROC) analysis. The index is defined for all points of an ROC curve, and the maximum value of the index may be used as a criterion for selecting the optimum cut-off point when a diagnostic test gives a numeric rather than a dichotomous result. In the present invention, the binary number is set as 1 when HbAlc>=6.5%. Accordingly, the GMM-index is −1.028883 when Youden's index reaches the maximum; and the ESP-index is 4.4 when Youden's index reaches the maximum. That is, if a subject is determined to have a GMM-index higher than −1.028883, he/she may have an HbAlc level lower than 6.5%, with the accuracy being 90.48%; if a subject is determined to have a GMM-index lower than or equal to −1.028883, he/she may have an HbAlc level higher than 6.5%, with the accuracy being 44.75%. For the ESP-index, if a subject is determined to have an ESP-index higher than 4.4, he/she may have an HbAlc level lower than 6.5%, with the accuracy being 92.11%; if a subject is determined to have an ESP-index lower than or equal to 4.4, he/she may have an HbAlc level higher than 6.5%, with the accuracy being 45.52%.

For the method of monitoring response to disease treatment or diet intervention in a subject having T2D, the subject is determined to response positively to the disease treatment or diet intervention when the GMM-index or the ESP-index is increased or becomes close to or higher than a predetermined level in some embodiments during the disease treatment or diet intervention. The predetermined level is preferred to be approximately −1.028883 for the GMM-index or approximately 4.4 for the ESP-index, which are determined based on the respective ROC curve and the Younden's index, as described above.

The instant application also provides a microbe, comprising one or more of a bacteria corresponding-CAG NO.1-15, wherein CAG NO.1-15 comprises nucleic acids set forth in SEQ ID NO.: 1-191, 192-326, 327-593, 594-835, 836-885, 886-960, 961-1097, 1098-1264, 1265-1433, 1434-1684, 1685-1833, 1834-1979, 1980-2163, 2164-2447, and 2448-2783 respectively.

### EXAMPLES

Patients and Methods

GUT2D Study

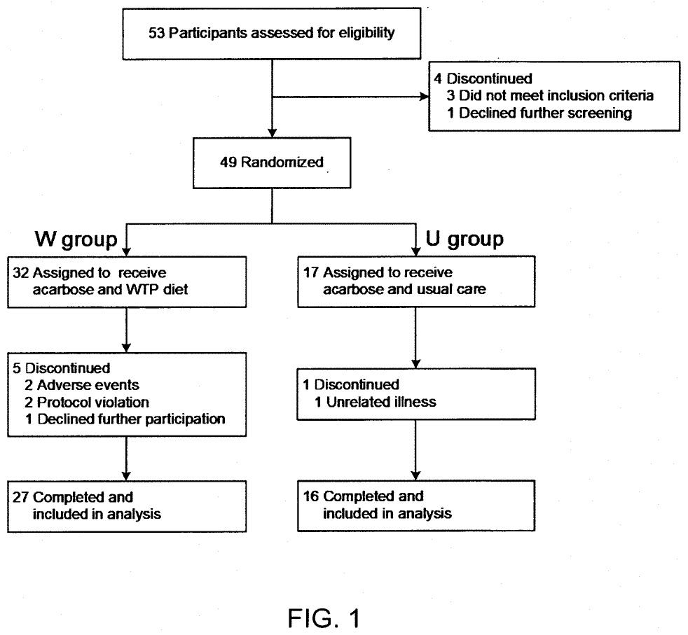

The randomized, open-label, parallel-group clinical trial for patients with type 2 diabetes mellitus (T2DM) was approved by the Ethics Committee at Shanghai General Hospital, Shanghai Jiao Tong University School of Medicine (No. 2014KY086), and the study was conducted in accordance with the principles of the Declaration of Helsinki. All participants provided written informed consent at the beginning of the trial. The trial was registered in the Chinese Clinical Trial Registry (No. ChiCTR-TRC-14004959). The design and progress of the clinical trial were shown in FIG. 1.

Recruited participants were 35-70-year-old Chinese Han patients with T2DM (6.5%≤HbAlc≤12.0%). The major exclusion criteria included: type 1 diabetes mellitus; pregnancy; lactation; an intent to become pregnant during the course of the study; severe diabetic complications (diabetic retinopathy, diabetic neuropathy, diabetic nephropathy and diabetic foot); severe hepatic diseases (including chronic persistent hepatitis, liver cirrhosis or the co-occurrence of positive hepatitis B virus surface antigen and abnormal hepatic transaminase (serum concentrations of alanine transaminase or aspartate transaminase >2.5× the upper limit of normal)); continuous antibiotic use for >3 days within 3 months prior to enrolment; continuous weight-loss drug use for >1 month; gastrointestinal surgery (except for appendicitis or hernia surgery); a severe mental illness in last 6 months; receiving drug therapy to treat cholecystitis, peptic ulcers, urinary tract infection, acute pyelonephritis, urocystitis or hyperthyreosis; pituitary dysfunction; severe organic diseases, including cancer, coronary heart disease, myocardial infarction or cerebral apoplexy; infectious diseases, including pulmonary tuberculosis and AIDS; and alcoholism.

During a 2-week run-in period, all antidiabetic drugs except for insulin secretagogues or insulin glargine were terminated to avoid potential effects of those drugs on the gut microbiota. Before the interventions (Day 0), all participants received health education about T2DM and a baseline evaluation. A meal-based food-frequency questionnaire and a 24-hour dietary record were used to calculate baseline nutrient intake based on China Food Composition 2009 (17). The participants were randomly assigned to receive acarbose plus usual care for T2DM (U group) or acarbose plus a diet formula (the WTP diet) based on whole grains, traditional Chinese medical foods and prebiotics (W group) for 84 days.

Usual care consisted of standard dietary and exercise advice according to Chinese diabetes guidelines for T2DM (2013 edition). The WTP diet included three ready-to-consume pre-prepared foods, Formula No. 1 (2), Formula No. 2 (2) and Formula No. 8 (manufactured by Perfect (China) Co. Zhongshan, China). For W group, the WTP diet was administered in combination with an appropriate amount of vegetables, fruits and nuts according to the dietician's advice. The intake of macronutrients was balanced according to standard nutritional requirements for age provided by the Chinese Dietary Reference Intakes (DRIs) and recommended by the Chinese Nutrition Society (CNS, 2013). Formula No. 1 was a pre-cooked mixture of 12 component materials from whole grains and traditional Chinese medicine (TCM) food plants that are rich in dietary fiber, including adlay (Coix lachrymal-jobi L.), oat, buckwheat, white bean, yellow corn, red bean, soybean, yam, peanut, lotus seed, and wolfberry, which was prepared in the form of canned gruel (370 g wet weight per can). Each contained 100 g of ingredients (59 g carbohydrate, 15 g protein, 5 g fat, and 6 g fiber) and 336 kcal (70% carbohydrate, 17% protein, 13% fat). Formula No. 2 was a powder preparation for infusion (20 g per bag) containing bitter melon (Momordica charantia) and oligosaccharides, including fructo-oligosaccharides and oligoisomaltoses. The detailed composition of Formula No. 8 is shown in Table 1 below. For each meal, ≥360 g of Formula No. 1 was consumed as the staple food, and Formulas No. 2 and No. 8 were consumed at 10 g and 15 g, respectively. The dietary record for each subject was used to calculate nutrient intake based on the China Food Composition 2009³⁹ (Table 2). Acarbose was administered using an oral dose of 100 mg, three times a day. Participants recorded their treatment regimens for diet, body weight, drug use and adverse events. Furthermore, self-monitored daily fasting blood glucose (FBG) and 2-hour postprandial blood glucose (2 h PBG) were recorded, and doses of background treatments (insulin secretagogues or insulin glargine) were adjusted according to improvements in symptoms and daily two-point glycaemic profiles (Table 3).

TABLE 1
The components of the ready-to-consume Formula No. 8 used in the WTP diet

| | Formula8^{a} | | Formula8^{a} |
|---|---|---|---|
| Ash content (g/100 g) | 0.45 | Inositol (mg/kg) | 90 |
| Water (g/100 g) | 80.5 | linoleic acid (g/100 g) | 0.28 |
| Protein (g/100 g) | 3.63 | α-linolenic acid (g/100 g) | 0.01 |
| Fat (g/100 g) | 1.2 | docosahexenoic acid (g/100 g) | / |
| Carbohydrate | 12.5 | eicosatetraenoic acid (g/100 g) | / |
| Fibre (g/100 g) | 1.7 | Cytidine (mg/100 g) | / |
| Soluble fibre (g/100 g) | 0.2 | Uridine (mg/100 g) | / |
| Insoluble fibre (g/100 g) | 1.6 | Carnine (mg/100 g) | / |
| Vitamin A (mg/kg) | / | Guanosine (mg/100 g) | / |
| Vitamin D (mg/kg) | / | Adenosine (mg/100 g) | / |
| Vitamin E (mg/kg) | 2.80 | Choline (mg/100 g) | 10 |
| Vitamin K1 (μg/100 g) | / | L-carnitine (mg/kg) | / |
| Vitamin B1 (mg/100 g) | / | Taurine (mg/100 g) | 1 |
| Vitamin B2 (mg/100 g) | 0.082 | Molybdenum (mg/kg) | 1 |
| Vitamin B6 (μg/100 g) | / | Cobalt (mg/kg) | 1 |
| Vitamin B12 (μg/100 g) | / | Aspartic acid (g/100 g) | 0.01 |
| Vitamin C (mg/100 g) | <0.3 | Threonine (g/100 g) | / |
| Biotin (μg/100 g) | / | Serine (g/100 g) | 0.01 |
| Niacin (μg/100 g) | 220 | Glutamic acid (g/100 g) | 0.01 |
| VitaminB5 (μg/100 g) | / | Proline (g/100 g) | / |
| Folate (μg/100 g) | 11.1 | Glycine (g/100 g) | 0.01 |
| Sodium (mg/kg) | 67 | Alanine (g/100 g) | 0.01 |
| Potassium (mg/kg) | 18000 | Valine (g/100 g) | 0.01 |
| Copper (mg/kg) | 1 | Cystine (g/100 g) | 0.01 |
| Magnesium (mg/kg) | 337 | Methionine (g/100 g) | 0.01 |
| Iron (mg/kg) | 9 | Isoleucine (g/100 g) | / |
| Zinc (mg/kg) | 5 | Leucine (g/100 g) | / |
| Manganese (mg/kg) | 5 | Tyrosine (g/100 g) | / |
| Calcium (mg/kg) | 158 | Phenylalanine (g/100 g) | / |
| Phosphorus (mg/100 g) | 74.2 | Histidine (g/100 g) | 0.01 |
| Iodine (mg/kg) | 0.12 | Trytophan (g/100 g) | 0.01 |
| Chlorine (mg/100 g) | 32.6 | Lysine (g/100 g) | / |
| Selenium (mg/kg) | 0.016 | Arginine (g/100 g) | / |
| Chromium (mg/kg) | / | Total amino acid | 0.1 |
| | | (g/100 g) | |
| Fluorine (mg/kg) | <0.5 | Energy (kJ/100 g) | 333 |

^{a}Ready-to consume dry powder.

TABLE 2
Daily energy and macronutrient intake before
and during the dietary intervention^{a}

| Group | Daily intake | Day 0 | Day 84 |
|---|---|---|---|
| W | Total Energy | 1924.93 ± 129.67 | 1874.87 ± 71.10 |
| | (kcal) | | |
| (N = | Fat (g) | 63.48 ± 4.57 | 58.32 ± 4.04 |
| 24) | Fat % | 31.03 ± 1.86 | 27.54 ± 1.07 |
| | Protein (g) | 81.52 ± 5.90 | 74.58 ± 3.67 |
| | Protein % | 16.94 ± 0.63 | 15.88 ± 0.49 |
| | Total | 268.77 ± 25.67 | 282.72 ± 9.63 |
| | carbohydrate | | |
| | (g) | | |
| | Total | 52.03 ± 2.16 | 56.58 ± 1.09 |
| | carbohydrate | | |
| | % | | |
| | Total | 12.12 ± 1.24 | 37.10 ± 1.90***^{###} |
| | fibre (g) | | |
| | Soluble | 4.59 ± 0.47 | 14.61 ± 0.69***^{###} |
| | fibre (g) | | |
| U | Total | 2063.54 ± 161.42 | 1954.48 ± 142.80 |
| | Energy | | |
| | (kcal) | | |
| (N = | Fat (g) | 70.44 ± 8.30 | 62.41 ± 5.14 |
| 14) | Fat % | 30.70 ± 2.39 | 29.16 ± 1.57 |
| | Protein (g) | 87.31 ± 9.14 | 79.32 ± 9.00 |
| | Protein % | 16.65 ± 0.88 | 15.76 ± 0.86 |
| | Total | 285.53 ± 24.85 | 284.94 ± 21.45 |
| | carbohydrate | | |
| | (g) | | |
| | Total | 52.65 ± 2.44 | 55.08 ± 1.63 |
| | carbohydrate | | |
| | % | | |
| | Total | 15.43 ± 2.43 | 16.06 ± 1.95 |
| | fibre (g) | | |
| | Soluble | 5.85 ± 0.92 | 6.09 ± 0.74 |
| | fibre (g) | | |

^{a}Data are means ± sem.
***P < 0.001 versus W Day 0;
^{###}P < 0.001 versus U Day 84.
Two-way repeated measures analysis of variance with the Bonferroni post hoc test was used for the intra- and inter-group comparisons.

TABLE 3
Antidiabetic Medication use^{a}

| Group | ID | trial | Drug usage (except acarbose) during the intervention |
|---|---|---|---|
| W | DBH1W001 | Repaglinide, 2 mg, tid | None |
| | DBH1W002 | None | None |
| | DBH1W003 | Glimepiride, 2 mg, qd | None |
| | DBH1W004 | None | None |
| | DBH1W005 | Glimepiride, 1 mg, qd | None |
| | DBH1W006 | None | None |
| | DBH1W007 | Insulin, 16U IH before | Insulin, I6U IH before breakfast, 15U IH before dinner, |
| | | breakfast, 15U IH before | day −14 to day 9; Insulin, 10U IH before breakfast, |
| | | dinner | 8U IH before dinner, day 10 to day 11; Insulin, |
| | | | 6U IH before breakfast, 6U IH before dinner, day 12 to |
| | | | day 15; Insulin, 6U IH before breakfast, day 16 to day 20; |
| | | | None, day 21 to the end of the intervention |
| | DBH2W002 | Insulin, 24U IH before | Insulin, 24U IH before breakfast, 16U IH before dinner, |
| | | breakfast, 16U IH before | day −14 to day 3; Insulin, 24U IH before breakfast, |
| | | dinner | 12U IH before dinner, day 4 to day 5; Insulin, |
| | | | 22U IH before breakfast, 12U IH before dinner, day 6 to |
| | | | day 14; Insulin, 20U IH before breakfast, 12U IH before |
| | | | dinner, day 15 to day 46; Insulin, 18U IH before breakfast, |
| | | | 10U IH before dinner, day 47 to day 51; Insulin, |
| | | | 16U IH before breakfast, 10U IH before dinner, |
| | | | day 47 to the end of the intervention |
| | DBH2W004 | Gliclazide, 80 mg, qd | Gliclazide, 80 mg, qd, day −14 to day 23; None, |
| | | | day 24 to the end of the intervention |
| | DBH2W006 | Gliclazide, 80 mg, qd | Gliclazide, 80 mg, qd day −14 to day 12; None, |
| | | | day 13 to the end of the intervention |
| | DBH2W007 | Gliquidone, 30 mg, qd | None |
| | DBH2W008 | Repaglinide, 2 mg, tid | Repaglinide, 2 mg, bid, day −14 to day 7; |
| | | | Repaglinide, 2 mg, qd, day 8 to day 9; |
| | | | None, day 10 to the end of the intervention |
| | DBH2W009 | Metformin, 500 mg, qd, | None |
| | | Repaglinide, 2 mg, qd | |
| | DBH2W011 | Glimepiride, 2 mg, qd | Glimepiride, 2 mg, qd, day −14 to day 10; |
| | | | None, day 11 to the end of the intervention |
| | DBH2W012 | None | None |
| | DBH2W013 | Metformin, 250 mg, bid; | Glipizide, 5 mg, qd, day −14 to day 17; |
| | | Glipizide, 5 mg, qd | None, day 18 to the end of the intervention |
| | DBH2W015 | Glimepiride, 2 mg, qd; | Glimepiride, 1 mg, qd |
| | DBH2W016 | Gliclazide, 160 mg, qd; | Gliclazide, 80 mg, qd |
| | | Acarbose, 50 mg, qd; | |
| | | Metformin, 500 mg, qd | |
| | DBH2W017 | Metformin, 500 mg, tid; | Glimepiride, 1 mg, bid |
| | | Glimepiride, 1 mg, bid | |
| | DBH2W018 | Glipizide, 5 mg, qd, | Glipizide, 5 mg, qd, day −14 to day 7; |
| | | | None, day 8 to the end of the intervention |
| | DBH2W019 | Metformin, 250 mg, qid; | Gliclazide, 240 mg, qd, day −14 to day 9; |
| | | Gliclazide, 240 mg, qd; | Gliclazide, 160 mg, qd, day 10 to day 15; |
| | | | Gliclazide, 80 mg, qd, day 16 to the end of the |
| | | | intervention |
| | DBH3W001 | Tang Niao Le (a kind of | None |
| | | Chinese patent medicine) | |
| | DBH3W002 | None | None |
| | DBH3W003 | None | None |
| | DBH3W004 | Metformin, 500 mg, tid | None |
| | DBH3W006 | None | None |
| | DBH3W007 | Gliclazide, 160 mg, qd | None |
| U | DBH2U001 | Gliclazide, 80 mg, bid | None |
| | DBH2U002 | Insulin, 16U IH before | Insulin, 12U IH before breakfast, |
| | | breakfast, I4U IH before | 10U IH before dinner |
| | | dinner | |
| | DBH2U003 | Metformin, 500 mg, tid | None |
| | DBH2U004 | Gliclazide, 80 mg, bid | Gliclazide, 80 mg, bid, day −14 to day 6); |
| | | | Gliclazide, 80 mg, qd, day 62 to the end of the |
| | | | intervention |
| | DBH2U006 | Gliclazide, 120 mg, tid | Gliclazide, 80 mg, bid, day −14 to day 30; |
| | | | Gliclazide, 80 mg, qd, day 31 to day 32; |
| | | | None, day 33 to the end of the intervention |
| | DBH2U007 | None | None |
| | DBH2U008 | Metformin, 500 mg, qd; | Gliclazide, 80 mg, qd |
| | | Gliclazide, 80 mg, qd | |
| | DBH2U009 | None | None |
| | DBH2U010 | Insulin, 10U IH before | Insulin, 10U IH before breakfast, |
| | | breakfast, 10U IH before | 10U IH before dinner, day −14 to day 7; |
| | | dinner | Insulin, 8U IH before breakfast, |
| | | | 8U IH before dinner, day 8 |
| | | | to the end of the intervention |
| | DBH2U011 | None | None |
| | DBH2U012 | None | None |
| | DBH2U013 | Repaglinide, 2 mg, qd | None |
| | DBH2U014 | Gliclazide, 160 mg, qd | Gliclazide, 160 mg, qd |
| | DBH2U015 | Insulin, 34U IH before | Insulin, 18U IH before breakfast, |
| | | breakfast, 22U IH before | 14U IH before dinner, day −14 to day 29; |
| | | dinner | Insulin, 20U IH before breakfast, |
| | | | 18U IH before dinner, day 30 |
| | | | to the end of the intervention |
| | DBH2U016 | Insulin, 22U IH before | Insulin, 22U IH before breakfast |
| | | breakfast | |
| | DBH3U001 | Metformin, 500 mg, qd; | Glimepiride, 2 mg, qd, day −14 to day 37; |
| | | Glimepiride, 2 mg, qd | None, day 38 to the end of the intervention |

^{a}The intervention began following a 2-week washout period of the above regular medication. Day −14 indicated the beginning of the washout period.

Biological samples, anthropometric data and clinical laboratory analysis were obtained at baseline and every 28 days during the intervention. Venous blood samples were collected after 10 h of overnight fasting, and participants then underwent a 3-h oral glucose tolerance test. All participants ingested 75 g of glucose, and blood samples were obtained at 30, 60, 120 and 180 min. Blood samples were centrifuged at 3,000×g for 20 min after standing at room temperature for 30 min, to obtain serum. Faeces and morning urine were collected on the same day. Serum, urine and faecal samples were collected, immediately transferred to dry ice and stored at −80° C. within 5 h for additional analysis.

Bioclinical parameters were determined at the Shanghai General Hospital, Shanghai Jiao Tong University School of Medicine, Shanghai, China.

QIDONG study

This clinical trial, conducted at the Qidong People's Hospital (Jiangsu, China), examined the effect of a high dietary fibre diet in free-living conditions in a cohort of healthy individuals, and those with prediabetes and clinically diagnosed T2DM (QIDONG; Chinese Clinical Trial Registry: ChiCTR-IPC-14005346). The baseline phenotypic characteristics of the T2DM sub-group were largely similar to those in GUT2D. Participants with T2DM were randomised to receive either the WTP diet (without acarbose; n=71) or usual care (n=33) for 84 days. Blood and faecal samples were collected at baseline and at the end of the intervention, in which HbAlc and gut microbial profile were determined respectively.

Statistical Analysis

Statistical analyses were conducted using the SPSS Statistics 17.0 Software Package (SPSS Inc., Chicago, USA). A two-way repeated measures analysis of variance with Tukey's post-hoc test (two-tailed) was used for intragroup and intergroup comparisons of the bioclinical parameters and inflammation-related markers, respectively. Pearson Chi-square tests (two-tailed) were used to analyse variations in gender and the proportion of participants whose HbAlc was below 7.0% or 6.5% in the two groups. A Mann-Whitney U test (two-tailed) was used to analyse variations in other characteristics between the two groups at baseline.

Gut Microbiota Transplantation

Faecal samples were collected from two female participants (2W009 from the W group and 2U004 from the U group) at Day 0 and Day 84. These two donors were selected systemically—changes in the gut microbial profile after the interventions were determined in all participants, those with non-significant changes were excluded, then one participant from each group was randomly selected as the representative donor. Each faecal sample (0.5 g) was diluted in 25 mL of a sterile Ringer working buffer (9 g/L of sodium chloride, 0.4 g/L of potassium chloride, 0.25 g/L of calcium chloride dihydrate and 0.05% (w/v) L-cysteine hydrochloride) in an anaerobic chamber (80% N2:10% CO2:10% H2). The faecal material was suspended by thorough vortexing (5 min) and settled by gravity for 5 min. The clarified supernatant was transferred to a clean tube, and an equal volume of 20% (w/v) skimmed milk (LP0031, Oxoid, UK) was added. The inoculum was freshly prepared on the day of experiment, with the rest stored at −80° C. until the second inoculation.

All animal experimental procedures were approved by the Institute of Zoology Institutional Animal Care and Use Committee of the Chinese Academy of Sciences and were conducted according to the committee's guidelines. Weaned, germ-free female C57BL/6J mice (n=30) were maintained in flexible-film plastic isolators under a regular 12-h light cycle (lights on at 06:00). Sampling of faeces, food, water and padding were collected before transplantation. Normal saline was added into the samples with sufficient mixing. The mixtures were then cultured using the spread plate method on: 1) LB agar, Brain Heart Infusion agar and Thioglycolate agar under aerobic condition at 37° C. for aerobic bacteria; 2) on Gifu anaerobic medium (GAM) agar under anaerobic condition at 37° C. for anaerobic bacteria; and 3) on Modified Martin Agar and Tryptone Soya agar under aerobic condition at 25-28° C. for fungi. All cultures were examined under optical microscope after 1, 2, 4, 7 and 14 days.

Mice were fed ad libitum with a sterile normal chow diet (SLAC, Shanghai China). Surveillance for bacterial contamination was performed by periodic bacteriological examinations of faeces, food and padding. At 6 weeks of age, the germ-free mice were housed in individual cages and randomly divided into four groups (each group was kept in an individual isolator). After 2 weeks of acclimation, the four groups of mice were oral gavaged with 100 μL of one of the following faecal suspension inoculum: 2W009 at Day 0 (W-Pre; n=10), 2W009 at Day 84 (W-Post; n=10), 2U004 at Day 0 (U-Pre; n=5) and 2U004 at Day 84 (U-Post; n=5). Inoculation was repeated on the next day to reinforce the microbiota transplantation. On Day 14, after 8 h of overnight fasting, all mice underwent a 2-h oral glucose tolerance test (OGTT). Following oral gavage of D-glucose (2 g/kg body weight), blood samples were collected from the tail vein at 0, 15, 30, 60, 90 and 120 min with glucose levels determined using a glucometer (Accu-Chek® Performa).

Gut Microbiota Analysis

1. Metagenomic sequencing DNA was extracted from faecal samples as previously described (2), and were sequenced using an Illumina HiSeq 3000 at GENEWIZ Co. (Beijing, China). Cluster generation, template hybridisation, isothermal amplification, linearisation, and blocking denaturing and hybridisation of the sequencing primers were performed according to the workflow specified by the service provider. Libraries were constructed with an insert size of approximately 500 bp followed by high-throughput sequencing to obtain paired-end reads with 150 bp in the forward and reverse directions.

2. Data quality control Prinseq (3) was employed to: 1) trim the reads from the 3′ end until reaching the first nucleotide with a quality threshold of 20; 2) remove read pairs when either read was <60 bp or contained “N” bases; and 3) de-duplicate the reads. Reads that could be aligned to the human genome (H. sapiens, UCSC hg19) were removed (aligned with Bowtie2 (4) using —reorder—no-hd—no-contain—dovetail (seed sequence set as 20 bp in length)).

3. De novo non-redundant metagenomic gene-catalogue construction and gene-abundance-profile calculations High-quality paired-end reads from each sample were used for de novo assembly with IDBA_UD (5) into contigs of at least 500 bp. Genes were predicted using MetaGeneMark (6). A non-redundant gene catalogue of 4,893,833 microbial genes was constructed with CH-HIT using the parameters “-c 0.95 -aS 0.9”. High quality reads were mapped onto the gene catalogue using SOAPaligner (7). Aligned results were sampled and downsized to 31 million per sample. The soap.coverage.script was used to calculate gene-length normalised base counts in each downsizing step. The sampling procedure was repeated 30 times, and the mean value of the abundance was used in further analyses.

4. Co-abundance gene groups (CAGs) A Canopy-based clustering algorithm (8) was used to bin all genes based on their abundance across all samples with default parameters. Raw CAGs were removed in the subsequent analyses: 1) genes that had a Spearman correlation <0.7 with the canopy profile; 2) 90% of the total canopy profile was distributed in no more than three samples; 3) CAGs with less than three genes. Large CAGs with >700 genes were regarded as bacterial CAGs for further analyses. The principal component analyses of the bacterial CAGs based on the Bray-Curtis distance and Procrustes were performed with QIIME (9).

5. Assembly and taxonomic assignment of bacterial CAGs De novo assembly for each of the 180 prevalent bacterial CAGs was performed as previously described (2). Briefly, the CAG- and sample-specific reads were achieved by aligning all high-quality reads to the CAG-specific contigs, followed by de novo assembly with Velvet (10). We adopted the six criteria for high-quality draft genome assembly from the Human Microbiome Project (HMP) (http://www.hmpdacc.org/reference_genomes/finishing.php) and checkM (11) to assess the quality of the assemblies: 1) 90% of the genome assembly must be included in contigs >500 bp; 2) 90% of the assembled bases must be at >5x reads coverage; 3) the contig N50 must be >5 kb; 4) scaffold N50 must be >20 kb; 5) average contig length must be >5 kb; and 6) >90% of the core genes must be present in the assembly. We used two methods to identify the phylogenetic taxonomy of the CAGs whose high-quality draft genomes met at least five HMP criteria. First, a phylogenetic tree was constructed with the 154 bacterial CAGs with high quality assemblies, 352 reference gastrointestinal tract genomes from the HMP DACC database and the server's inbuilt database using the CVtree3.0 web server (12), which applies a composition vector to perform phylogenetic analysis. Then we also applied SpecI (13), which is a method to group organisms into species clusters based on 40 universal and single-copy phylogenetic marker genes, to delineate the bacterial CAGs. CAGs of low quality were aligned to the 7,991 reference genomes from the NCBI database at both the protein (BLASTP) and nucleotide (BLASTN) levels. The alignments were filtered with query coverage (>70%) and the E-value (<1e-10 at the nucleotide and <1e-5 at the protein level). Based on the taxonomic assignment threshold that was previously described (14), the CAGs were assigned to the species or genus levels (species level: 90% of genes can be mapped to the species' genome with >95% identity at the DNA level; genus level: 80% of genes can be mapped to a genus with >85% identity at both the DNA and protein levels).

6. GMM-Index and ESP-Index Calculation

The high-quality reads from each sample of the GUT2D and/or QIDONG dataset were aligned to the 64 high quality draft genomes with Bowtie2 with the parameters —reorder—no-hd—no-contain—dovetail (seed sequence set to be 20 bp in length). The alignments with YT:Z:DP (indicates the read was part of a pair and the pair aligned discordantly) were filtered. GMM-index=log (Σᵢ₌₁¹⁵Aᵢ/Σᵢ₌₁₆⁶⁴Aᵢ), wherein, Aᵢ (abundance of CAG No: i)=number of reads aligned to CAG No.: i/(size of CAG No.: i×number of total reads). ESP-Index=ln (Heip×10¹⁰×Σᵢ₌₁¹⁵Aᵢ) , wherein Heip=(e^{H}−1)/14, H=−Σᵢ₌₁¹⁵AᵢlnAᵢ, Aᵢ (abundance of CAG No.: i)=number of reads aligned to CAG No.: i/(size of No.: i×number of total reads).

7. Statistical Analysis Intervention-responsive bacterial CAGs were identified using Wilcoxon matched-pair signed-rank tests (two-tailed) with adjustments according to Benjamini & Hochberg (18). The P value adjustment was performed in MATLAB® programs with the “mafdr” command. Random Forest analyses were performed with the R package “randomForest”, and cross-validation was performed with “rfcv”.

8. Data Availability

The raw pyrosequencing and Illumina read data for all samples have been deposited in the European Nucleotide Archive (ENA) under accession number of PRJEB1455 (GUT2D Study) and PRJEB15179 (QIDONG Study).

### Example 1 A High-Fibre Intervention Significantly Improves Bioclinical Parameters in Patients with T2DM

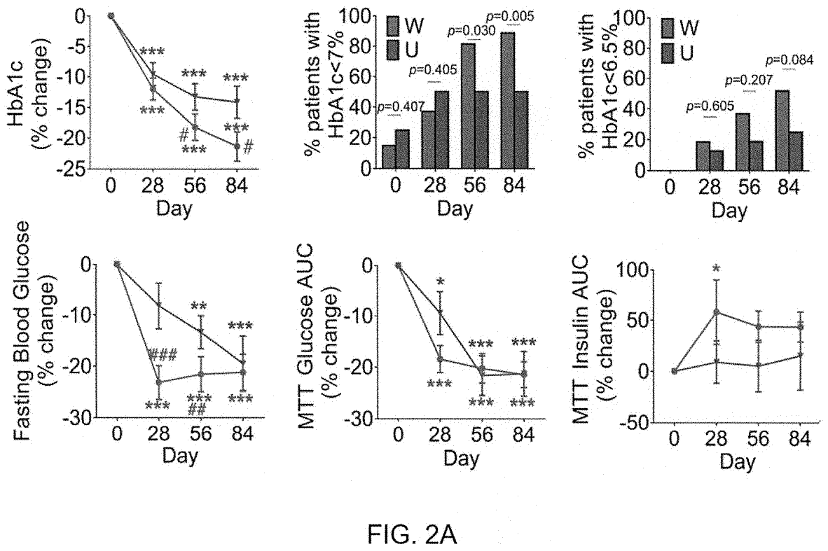

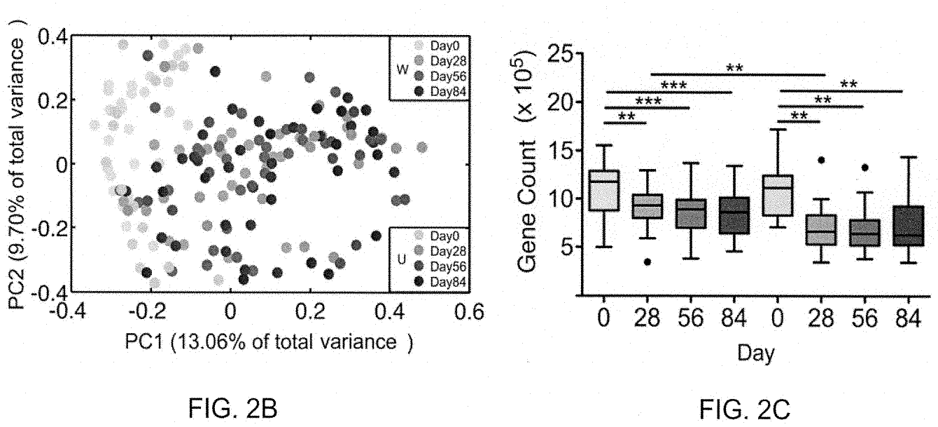

Almost all bioclinical parameters improved in both the W and U groups during the first month of the intervention. The level of glycated haemoglobin (HbAlc), the primary outcome in the current clinical trial, decreased significantly over time from baseline levels in both groups (FIG. 2A). By Day 84, reductions in HbAlc were greater in the W group than in the U group. At the end of the intervention (Day 84), the adequate-glycaemic-control rate (the proportion in the cohort with HbAlc <7%) was significantly higher in the W group than in the U group (88.9% versus 50.5%, P=0.005). The more stringent goal-achievement rate (the proportion of the cohort with HbAlc <6.5%) showed a similar (although non-significant) trend (51.9% versus 25.0%, P=0.084). Patients in the W group also lost a significantly greater percentage of body weight and demonstrated significantly improved lipid profiles and inflammation levels, compared with the U group. Levels of glucagon-like peptide-1 (GLP-1) and peptide YY (PYY), which can stimulate insulin secretion and inhibit glucagon secretion, increased significantly over time in the W group but not in the U group.

### Example 2 High-Fibre Interventions Modulate the Global Structure of the Gut Microbiota in Patients with T2DM

Shotgun metagenomic sequencing was performed on 172 faecal samples collected at 4 time points (Days 0, 28, 56 and 84). From a non-redundant gene catalogue of 4,893,833 microbial genes, 422 co-abundance gene groups (CAGs; binned using a Canopy-based algorithm (19)) were identified as distinct bacterial genomes. Based on Bray-Curtis distances from the 422 bacterial CAGs, the overall structure of the gut microbiota (as indicated by principal co-ordinate analysis) showed significant alteration from Day 0 to Day 28 in both groups with no further changes afterwards (FIG. 2B). At the end of the intervention (Day 84), significant difference (P=0.0056) in the gut microbial structure between the W and U groups reflected a distinct modulatory effect of the WTP diet on the gut microbiota. There was a notable reduction in gene richness (the number of genes identified per sample) in both groups, which followed a similar trend as in the overall microbial structure, i.e. significant reductions at Day 28 and remained stable for the rest of the intervention (FIG. 2C). This overall reduction of gene richness challenges the current notion that higher diversity implies better health (20). However, the gene richness at Day 28 was significantly higher in the W as compared to the U group and a similar trend was observed at Day 56 and Day 84 (FIG. 2C), consistent with better metabolic outcomes in the W group. Further, a Procrustes analysis with all bioclinical variables combined and the 422 bacterial CAGs showed that structural changes in the gut microbiota were associated with improvements in the clinical outcomes during the intervention (P <0.0001 from 999 Monte-Carlo simulations). Taken together, it was showed that the WTP diet induced significant changes in the global structure of the gut microbiota and these were correlated with improved overall clinical outcomes in patients with T2DM.

### Example 3 Transplantation Indicates a Causal Contribution of the Gut Microbiota to Alleviation of T2DM

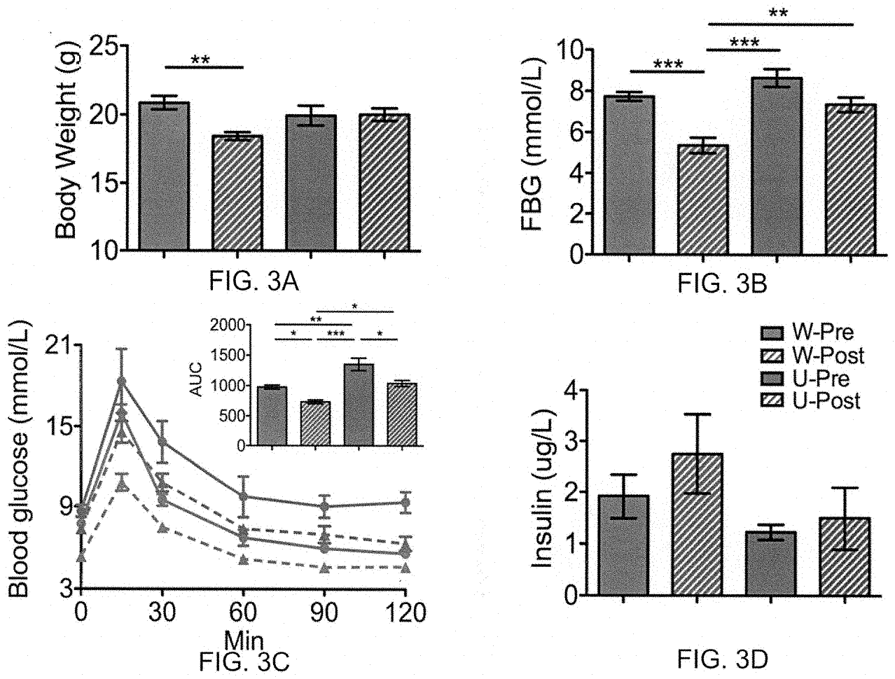

To establish causality between diet-altered gut microbiota and improvements in glucose metabolism, the pre- and post-intervention (Day 0 and Day 84 respectively) gut microbiota from participants in the W and U groups were transplanted into germ-free C57BL/6J mice. After 14 days of transplantation, mice receiving the post-intervention microbiota from the W group had a significantly lower body weight (FIG. 3A). These mice also had the lowest fasting and postprandial blood glucose when compared to those that were transplanted with the pre-intervention microbiota from the W group or the microbiota from the U group at either time points, an effect appeared to be associated with fasting insulin levels (FIG. 3B-D). The transferable effect of our interventions via microbial transplantation confirms that the high dietary fibre-induced changes in the gut microbiota causatively contribute to improved glucose homeostasis in patients with T2DM.

### Example 4 Specific Strains Respond to Fibre Intake

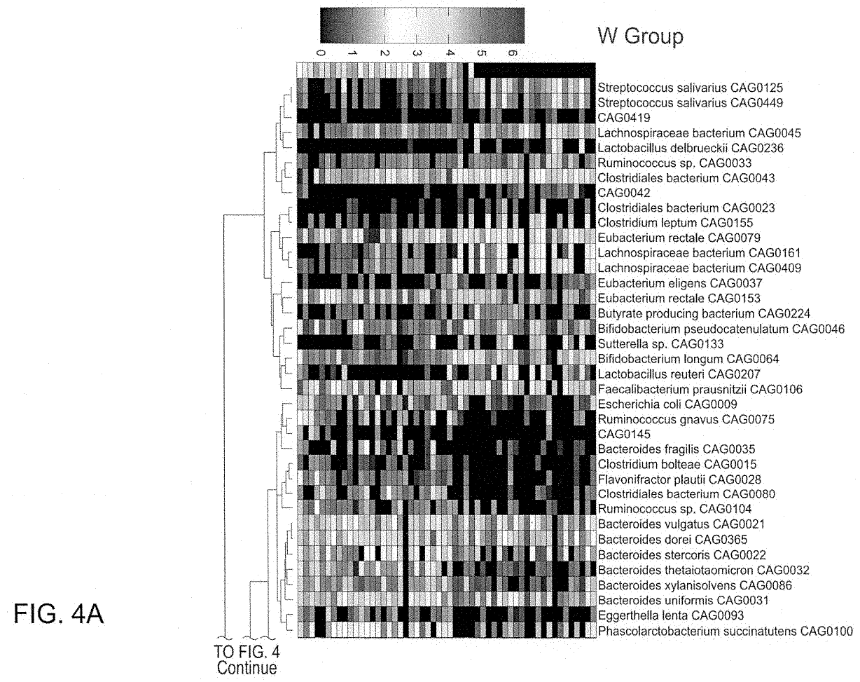

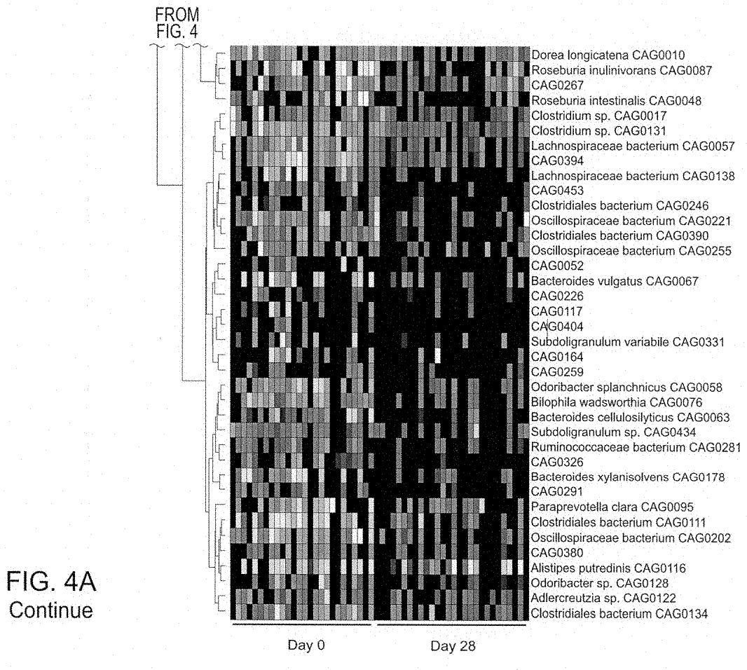

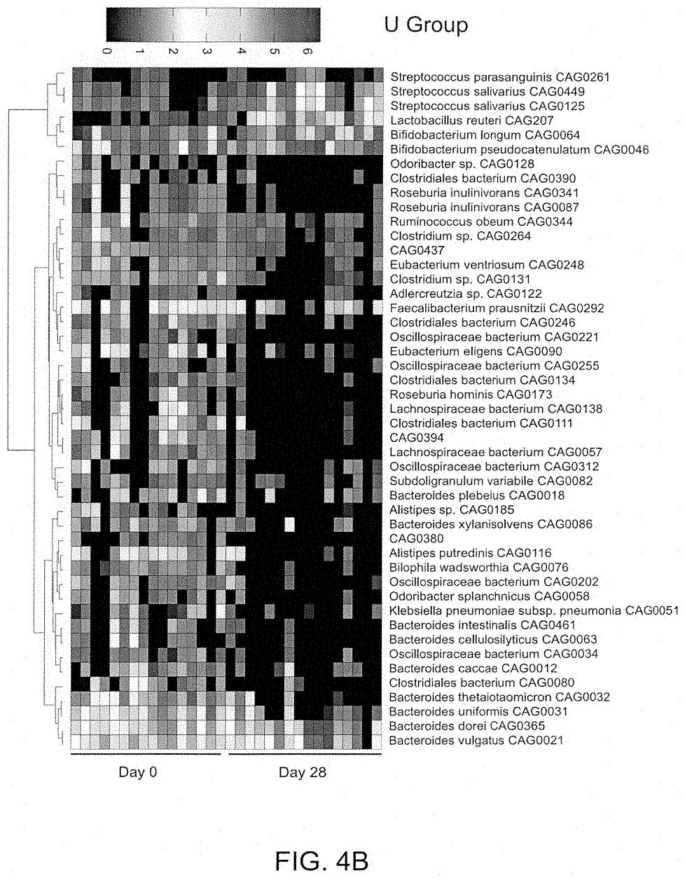

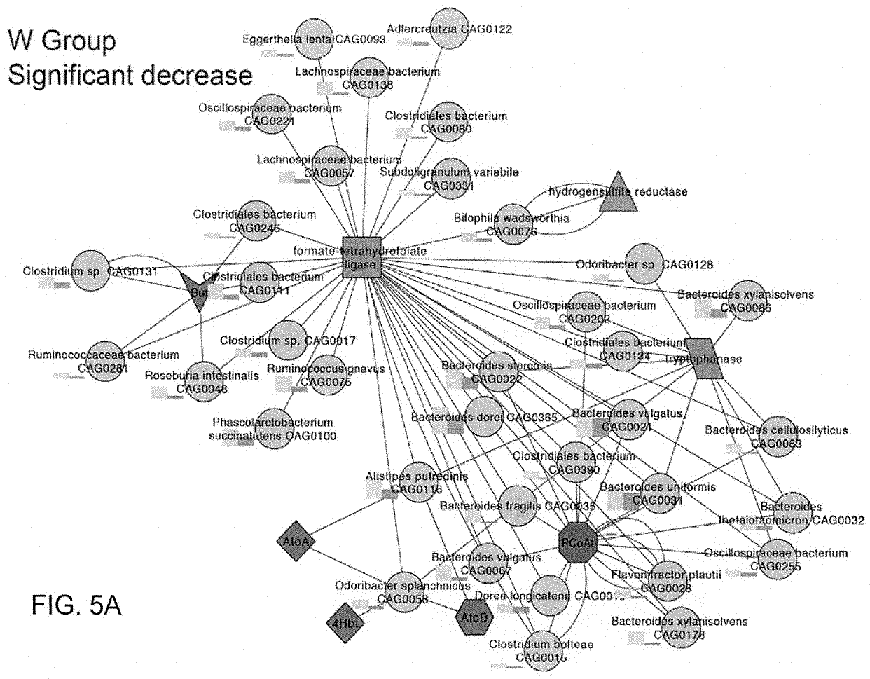

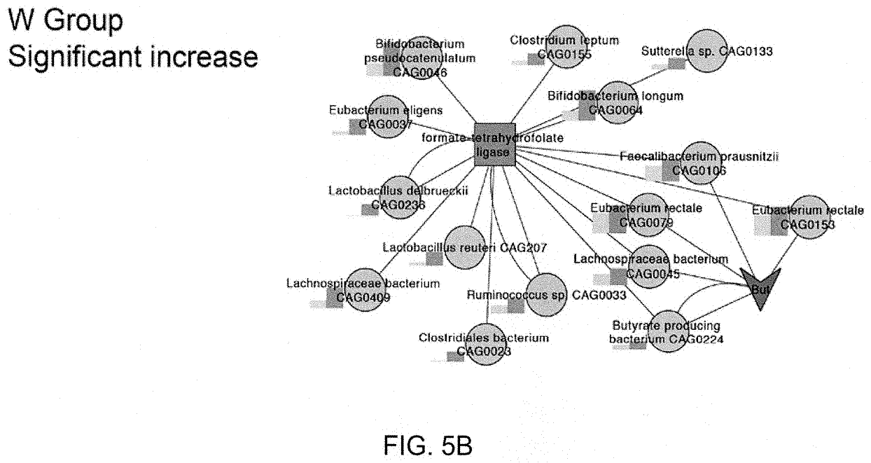

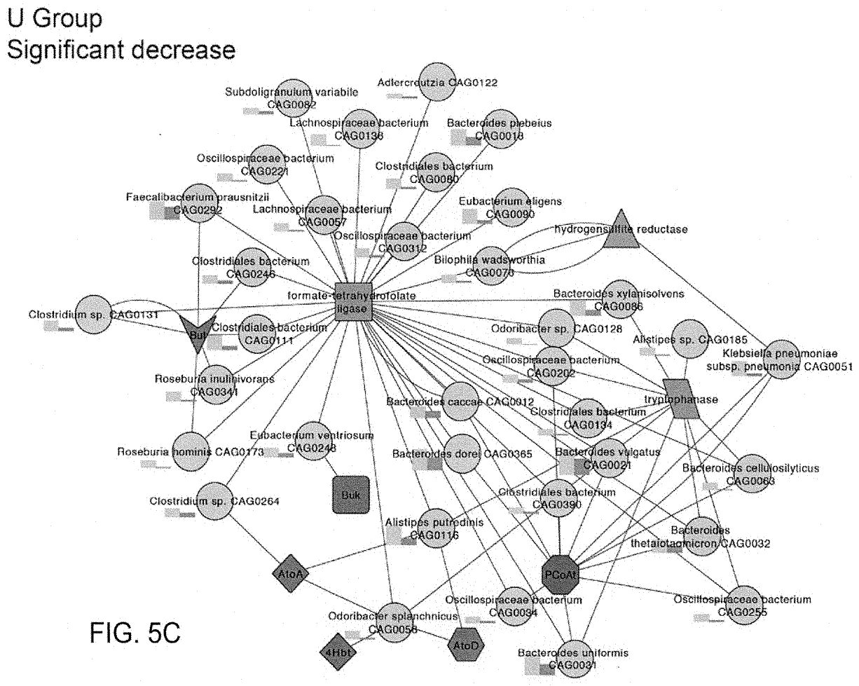

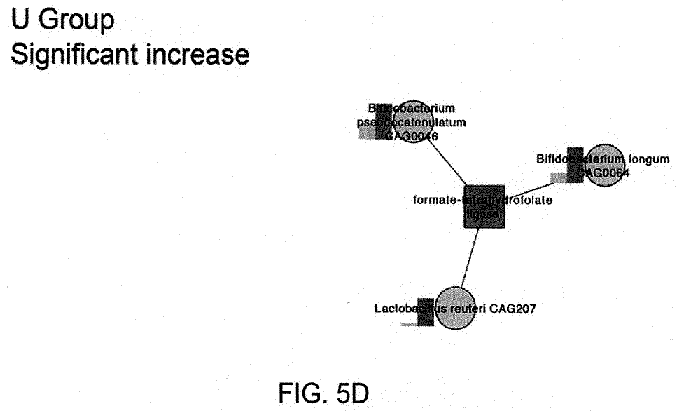

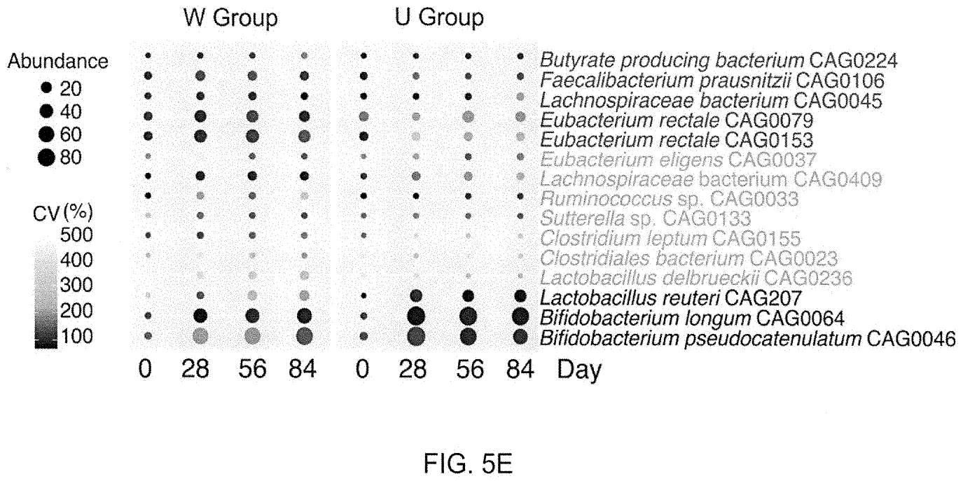

High-quality draft genomes were assembled to identify the bacterial species/strains that drive the gut-specific effects of dietary fibre on alleviating the T2DM phenotype. One hundred and fifty-four high-quality draft genomes were assembled from CAGs that were shared by >20% of the samples. The percentage of total reads per sample that was mapped to these high-quality draft genomes was 57% (±11%), which represented both the prevalent and dominant gut bacteria in the entire cohort. 141 of the 154 high quality draft genomes harbor at least one of the key genes for SCFA production, and can be considered as SCFA producers. Out of the 154 high-quality draft genomes, 64 bacteria were selected for further analysis because: 1) they are the intervention-responsive CAGs identified by Wilcoxon matched-pair signed-rank tests as significantly altered by the intervention at Day 28 in W or U group (FIG. 4); and 2) they harbor at least one of the genes for SCFA, H₂S or indole biosynthesis. All 15 genomes promoted in the W group harbor at least one of the genes for SCFA biosynthesis and genes for acetic acid production (including the 3 that were also enriched in the U group) and 5 of them also possess the capacity for butyrate biosynthesis (FIGS. 5B and 5D). This is consistent with the largely similar increase in faecal acetate and the enrichment of the acetic acid synthetic pathway in both groups but a distinct effect of the WTP diet on inducing butyrate production. The enrichment of these 15 genomes mostly peaked at Day 28 (FIG. 5E) that also accord with the pattern we observed in the overall gut microbiota, which further support these bacterial strains as the key drivers of structural shifts in the ecosystem.

These 15 bacteria, including Bifidobacterium spp., Lactobacillus spp., Eubacterium spp. and Faecalibacterium prausnitzii may serve the important purpose of replenishing acetate and butyrate in the W group and thus are likely the ecosystem service providers (ESPs) for that essential function. Efficient energy production from carbohydrates and tolerance to low pH may explain why these bacteria had a competitive edge over the other SCFA producers. A good example here is Bifidobacterium spp. which, taking advantage of its “bifid-shunt” pathway (21), is able to produce more ATP molecules and acetic acid comparing to other acetate producers. Intriguingly, despite the increase in the overall genetic capacity for SCFA production, most of SCFA producers were significantly diminished by our interventions (FIGS. 5A and 5C) that clearly suggest that not all bacteria that possess the functional genes can respond to substrate supplementation and become a provider of the function that the host needs. We envisage that this is at least partly driven by changes in gut luminal pH as some SCFA producers are known to be highly pH-sensitive, such as Bacteroides thetaiotaomicron and B. vulgatus (12). Accordingly, our data challenge the consensus in the microbiome field that assumes physiological relevance of gut bacteria to the host primarily based on gene-based functional predictions.

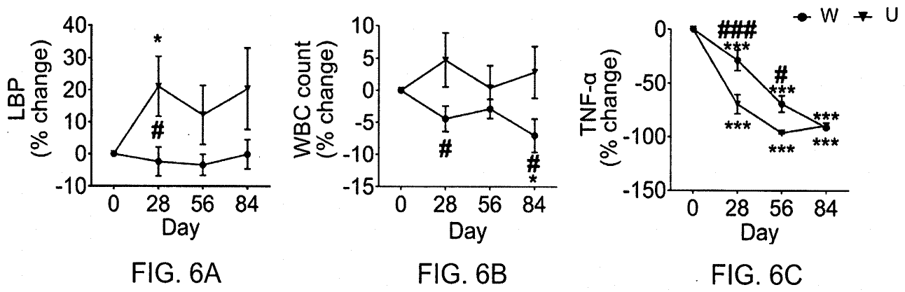

Among the 49 bacteria that were significantly down-regulated in either of the two groups were those that harbor genes for synthesising lipopolysaccharides, indole and H₂S. Again, in accordance with the gene-centric pathway analysis, this indicates that the reduced capacity for producing metabolically detrimental compounds is likely to contribute to the beneficial effects of the high dietary fibre diet. Reduced endotoxin production has been shown to alleviate inflammation and restore insulin sensitivity (22, 23). Lipopolysaccharide binding protein, the surrogate marker for endotoxin load, and inflammatory markers were lower in W than the U group, indicating the alleviation of inflammation probably due to reduced endotoxin production (FIG. 6). Decreased abundance of indole- and H₂S-producing bacteria ameliorate the inhibition on GLP-1 production (24-26), which accords with greater postprandial GLP-1 response observed in the W group. Taken together, it was showed that diminishing bacteria that produce detrimental metabolites lead to clinically significant improvements in the hosts.

The 15 ESPs mentioned above, CAG0023, CAG0033, CAG0037, CAG0045, CAG0046, CAG0064, CAG0079, CAG0106, CAG0133, CAG0153, CAG0155, CAG0207, CAG0224, CAG0236 and CAG0409, were designated as CAG NO.: 1 to 15, respectively, in the present invention. The 49 bacteria that were significantly downregulated, CAG0010, CAG0012, CAG0015, CAG0017, CAG0018, CAG0021, CAG0022, CAG0028, CAG0031, CAG0032, CAG0034, CAG0035, CAG0048, CAG0051, CAG0057, CAG0058, CAG0063, CAG0067, CAG0075, CAG0076, CAG0080, CAG0082, CAG0086, CAG0090, CAG0093, CAG0100, CAG0111, CAG0116, CAG0122, CAG0128, CAG0131, CAG0134, CAG0138, CAG0173, CAG0178, CAG0185, CAG0202, CAG0221, CAG0246, CAG0248, CAG0255, CAG0264, CAG0281, CAG0292, CAG0312, CAG0331, CAG0341, CAG0365, and CAG0390, were designated as CAG NO.: 16 to 64, respectively, in the present invention.

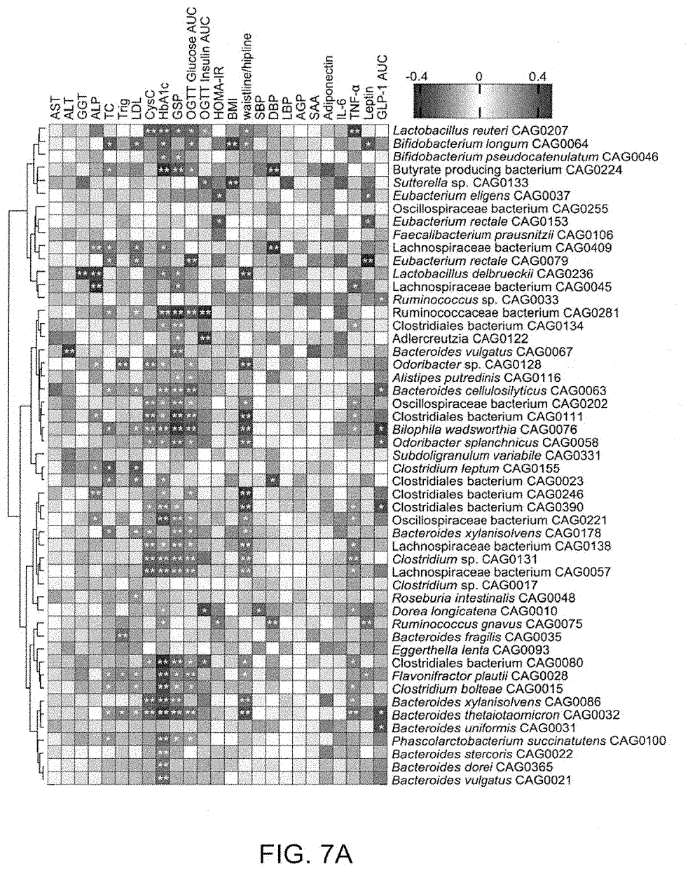

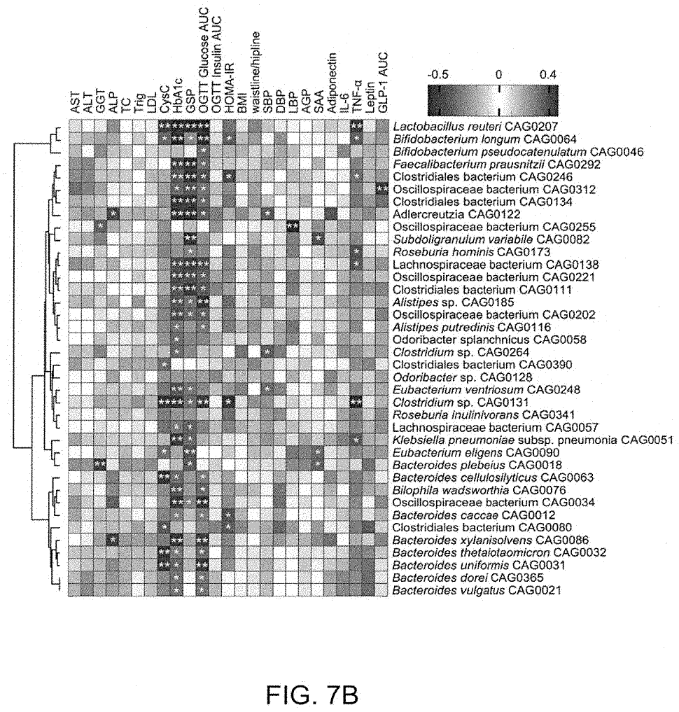

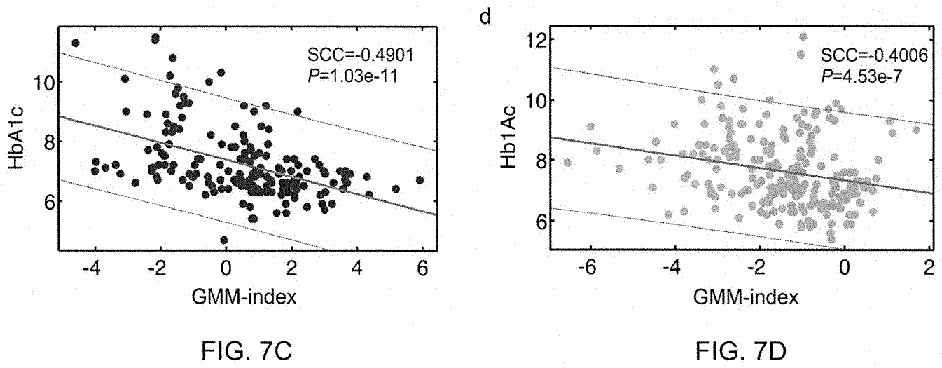

A Gut Microbiota Modulation (GMM)-index for each sample was calculated based on the abundance data of the 15 ESPs and also the 49 that decreased following intervention. GMM-index=log (Σᵢ₌₁¹⁵Aᵢ/Σᵢ₌₁₆⁶⁴Aᵢ), wherein, Aᵢ (abundance of CAG No: i)=number of reads aligned to CAG No.: i/(size of CAG No.: i×number of total reads). This GMM-index was significantly negatively correlated with the post-intervention level of HbAlc across all patients (Spearman correlation coefficient (SCC)=−0.4901, P=1.0253e⁻¹¹), indicating that shifts in the composition of the contributory bacteria in the microbiota, prompted by increased MACs, were associated with the primary clinical outcome (FIG. 7C).

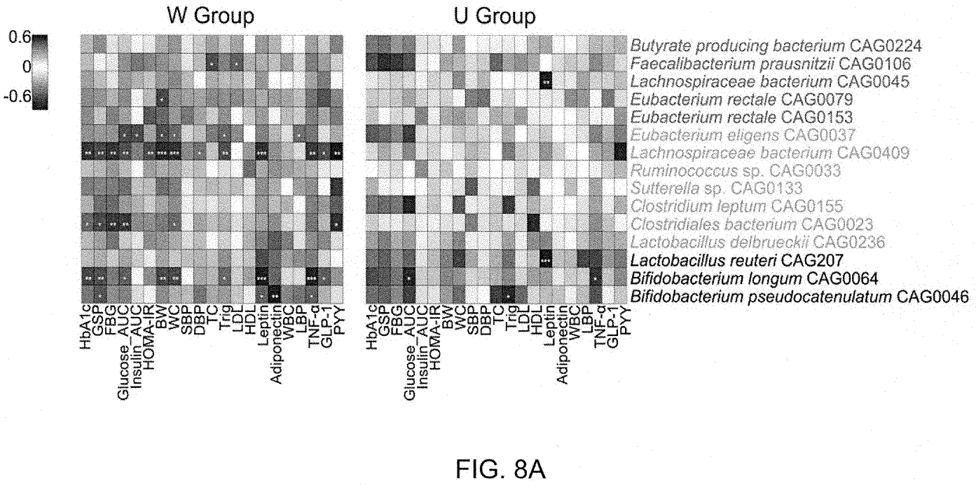

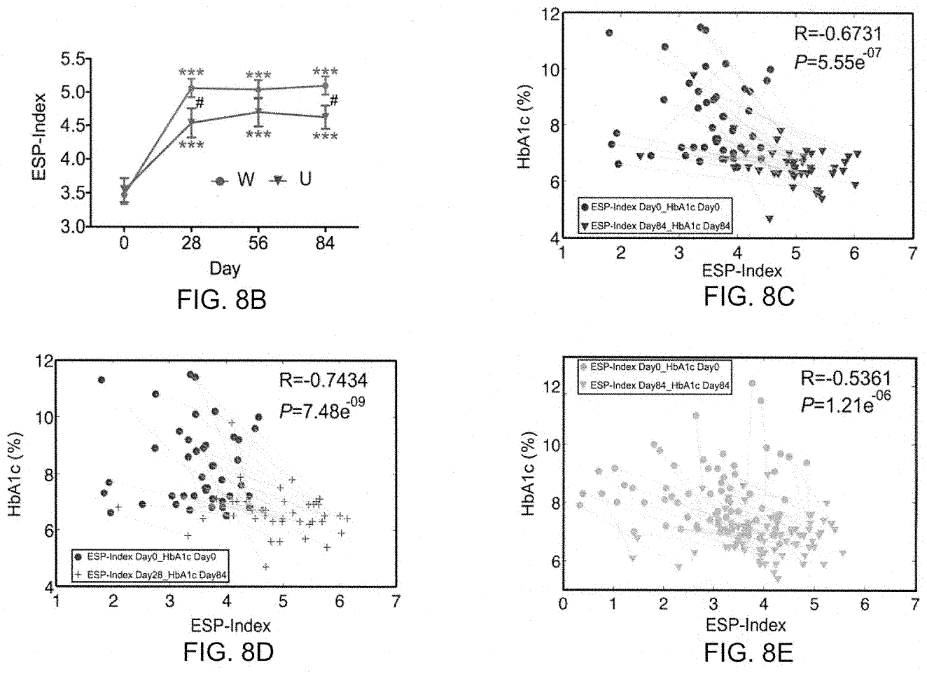

An ESP (ecosystem service provider)-index was calculated based on the abundance data of the 15 ESPs only that increased following intervention. ESP-Index=ln (Heip×10¹⁰×Σᵢ₌₁¹⁵Aᵢ), wherein Heip=(e^{H}−1)/14, H=−Σᵢ₌₁¹⁵AᵢlnAᵢ, Aᵢ (abundance of CAG No.: i)=number of reads aligned to CAG No.: i/(size of No.: i×number of total reads). The ESP-index followed a similar trajectory in both the W and U groups, i.e., a dramatic increase from baseline to Day 28 and remained at a similar level for the rest of the intervention, but the index was significantly higher in the W group at each of the post-intervention time points (Days 28, 56 and 84; FIG. 8B). The significant negative correlation between HbAlc and ESP-index at baseline and at the end of intervention (Day 0 and Day 84; r=−0.6731; P=5.55e⁻⁰⁷; FIG. 8C) confirmed the role of these ESPs in regulating host glucose homeostasis. While clinical outcomes such as HbAlc continued to decrease over the duration of intervention (FIG. 2A), the ESP-index plateaued from Day 28 onwards (FIG. 8B). Our data clearly indicate that dietary fibre-induced enrichment of the ESPs preceded significant changes in clinical outcomes. When ESP-index at Day 28 (instead of Day 84) was used with HbAlc at Day 84 to plot the post-intervention data points while keeping the exact same set of baseline data points as in FIG. 8C, a similar negative correlation was observed between HbAlc and ESP-index (r=−7434; P=7.485⁻⁰⁸; FIG. 8D). This suggests that ESP-index at Day 28, indicating the enrichment of the 15 ESPs at this early time point, may be informative on the eventual treatment outcomes that occur much later.

### Example 5 The Ecosystem Service Providers are Shared by Different T2DM Patient Cohorts

Finally, to find out whether the ecosystem service providers identified in GUT2D trial are shared by other T2DM patient cohorts, another independent clinical trial (QIDONG) was conducted in which 74 patients with T2DM received the WTP diet without acarbose for 3 months. Levels of HbAlc improved significantly from baseline after the intervention. Fecal samples were collected at baseline and end of each month for all the patients. 148 samples were metagenomically sequenced at an average depth of 14.1G. More than half of the sequenced reads were mapped onto the 154 high-quality draft genomes that were assembled in the GUT2DM project, showing that the corresponding prevalent gut bacteria were common to different cohorts of Chinese patients with T2DM. The 15 ESPs and the 49 bacteria that were co-excluded by promotion of these ESPs identified in GUT2D were present in patients of the QIDONG trial. Notably, using the second trial (without acarbose) to provide a test dataset, the GMM-index based on the 15 ESPs and their co-excluding bacteria had a similar significant negative correlation with the primary outcome (the level of HbAlc) (FIG. 7D).

Further, using the same set of 15 SCFA providers that were identified as positively responsive to dietary fibre in GUT2D, there was a similar negative correlation between the ESP-index and HbAlc in this QiDong intervention group (FIG. 8E).

Receiver operating characteristic curves (ROC) were built according to GMM-indexes from the 172 faecal samples collected in GUT2D study and the 148 samples collected in QIDONG study, with the leave-one-out cross-validation area under ROC (AUC) achieved 0.7052, wherein the binary number was set as 1 when HbAlc>=6.5%, and the specificity and sensitivity were 90.48% and 44.75%, respectively. The GMM-index was −1.028883 when Youden's index reached the maximum.

Further, receiver operating characteristic curves (ROC) were built according to ESP-indexes from the 172 faecal samples collected in GUT2D study, with the leave-one-out cross-validation area under ROC (AUC). achieved 0.70, wherein the binary number was set as 1 when HbAlc>=0.65%, and the specificity and sensitivity were 92.11% and 45.52%, respectively. The ESP-index was 4.4 when Youden's index reaches the maximum.

### REFERENCES

1. E. K. Costello, K. Stagaman, L. Dethlefsen, B. J. Bohannan, D. A. Relman, The application of ecological theory toward an understanding of the human microbiome. Science 336, 1255-1262 (2012).
2. A. Koh, F. De Vadder, P. Kovatcheva-Datchary, F. Backhed, From Dietary Fiber to Host Physiology: Short-Chain Fatty Acids as Key Bacterial Metabolites. Cell 165, 1332-1345 (2016).
3. J. Qin et al., A metagenome-wide association study of gut microbiota in type 2 diabetes. Nature 490, 55-60 (2012).
4. F. H. Karlsson et al., Gut metagenome in European women with normal, impaired and diabetic glucose control. Nature 498, 99-103 (2013).
5. K. Forslund et al., Disentangling type 2 diabetes and metformin treatment signatures in the human gut microbiota. Nature 528, 262-266 (2015).
6. N. Larsen et al., Gut microbiota in human adults with type 2 diabetes differs from non-diabetic adults. PloS one 5, e9085 (2010).
7. A. Soare et al., The effect of the macrobiotic Ma-Pi 2 diet vs. the recommended diet in the management of type 2 diabetes: the randomized controlled MADIAB trial. Nutrition & metabolism 11, 39 (2014).
8. M. Chandalia et al., Beneficial effects of high dietary fiber intake in patients with type 2 diabetes mellitus. The New England journal of medicine 342, 1392-1398 (2000).
9. F. M. Silva, C. K. Kramer, D. Crispim, M. J. Azevedo, A high-glycemic index, low-fiber breakfast affects the postprandial plasma glucose, insulin, and ghrelin responses of patients with type 2 diabetes in a randomized clinical trial. The Journal of nutrition 145, 736-741 (2015).
10. T. Chen et al., Fiber-utilizing capacity varies in Prevotella- versus Bacteroides-dominated gut microbiota. Sci Rep 7, 2594 (2017).

011. H. J. Flint, S. H. Duncan, K. P. Scott, P. Louis, Links between diet, gut microbiota composition and gut metabolism. Proc Nutr Soc 74, 13-22 (2015).
12. S. H. Duncan, P. Louis, J. M. Thomson, H. J. Flint, The role of pH in determining the species composition of the human colonic microbiota. Environmental microbiology 11, 2112-2122 (2009).
13. H. J. Flint, K. P. Scott, P. Louis, S. H. Duncan, The role of the gut microbiota in nutrition and health. Nature reviews. Gastroenterology & hepatology 9, 577-589 (2012).
14. G. Wu et al., Genomic Microdiversity of Bifidobacterium pseudocatenulatum Underlying Differential Strain-Level Responses to Dietary Carbohydrate Intervention. mBio 8, (2017).
15. C. Zhang et al., Dietary Modulation of Gut Microbiota Contributes to Alleviation of Both Genetic and Simple Obesity in Children. EBioMedicine 2, 966-982 (2015).
16. J. M. Bland, D. G. Altman, Calculating correlation coefficients with repeated observations: Part 1—Correlation within subjects. BMJ 310, 446 (1995).
17. Yuexin Yang, G. W., Xingchang Pang. China Food Composition (Book 1.2nd Edition). (Beijing Medical University Press, 2009).
18. P. D. Cani et al., Gut microbiota fermentation of prebiotics increases satietogenic and incretin gut peptide production with consequences for appetite sensation and glucose response after a meal. The American journal of clinical nutrition 90, 1236-1243 (2009).
19. H. B. Nielsen et al., Identification and assembly of genomes and genetic elements in complex metagenomic samples without using reference genomes. Nature biotechnology 32, 822-828 (2014).
20. E. Le Chatelier et al., Richness of human gut microbiome correlates with metabolic markers. Nature 500, 541-546 (2013).
21. K. Pokusaeva, G. F. Fitzgerald, D. van Sinderen, Carbohydrate metabolism in Bifidobacteria. Genes Nutr 6, 285-306 (2011).
22. L. Sun et al., A marker of endotoxemia is associated with obesity and related metabolic disorders in apparently healthy Chinese. Diabetes care 33, 1925-1932 (2010).
23. P. D. Cani et al., Metabolic endotoxemia initiates obesity and insulin resistance. Diabetes 56, 1761-1772 (2007).
24. M. T. Yokoyama, J. R. Carlson, Microbial metabolites of tryptophan in the intestinal tract with special reference to skatole. The American journal of clinical nutrition 32, 173-178 (1979).
25. C. Chimerel et al., Bacterial metabolite indole modulates incretin secretion from intestinal enteroendocrine L cells. Cell reports 9, 1202-1208 (2014).
26. V. Bala et al., Release of GLP-1 and PYY in response to the activation of G protein-coupled bile acid receptor TGRS is mediated by Epac/PLC-epsilon pathway and modulated by endogenous H2S. Frontiers in physiology 5, 420 (2014).

We claim:

1. A method for evaluating efficacy of diet intervention or disease treatment in a subject having type 2 diabetes mellitus, comprising method 1) or method 2),
wherein the method 1) comprising the steps of
a) collecting a fecal sample from the subject before and during the diet intervention or disease treatment;
b) analyzing DNA extracted from the fecal sample to determine abundance of each reference CAG selected from the group consisting of CAG ID Nos.: 1-64,
Aᵢ (abundance of CAG No: i)=number of reads aligned to CAG No.: i/(size of CAG No.: i×number of total reads;
d) calculating GMM-index of each sample using the calculated abundance data,
GMM-index=log (Σᵢ₌₁¹⁵Aᵢ/Σᵢ₌₁₆⁶⁴Aᵢ); and
e) determining that the subject responds positively to the diet intervention or disease treatment if the GMM-index is increased in the sample collected during the diet intervention or disease treatment,
wherein, CAG NOs.:1-15 comprise nucleic acid sequences set forth in SEQ ID NOs.: 1-191, 192-326, 327-593, 594-835, 836-885, 886-960, 961-1097, 1098-1264, 1265-1433, 1434-1684, 1685-1833, 1834-1979, 1980-2163, 2164-2447, and 2448-2783, respectively, and CAG NOs.:16-64 comprise nucleic acid sequences set forth in SEQ ID NOs.: 2784-2961, 2962-3130, 3131-3525, 3526-3747, 3748-3863, 3864-4068, 4069-4212, 4213-4393, 4394-4532, 4533-4891, 4892-4979, 4980-5116, 5117-5320, 5321-5464, 5465-5781, 5782-6279, 6280-6646, 6647-6954, 6955-7178, 7179-7613, 7614-7758, 7759-8046, 8047-8491, 8492-8546, 8547-9971, 9972-10099, 10100-10392, 10393-10502, 10503-10694, 10695-10986, 10987-11089, 11090-11262, 11263-11466, 11467-11704, 11705-12034, 12035-12113, 12114-12341, 12342-12454, 12455-12664, 12665-12825, 12826-13042, 13403-13500, 13501-13726, 13727-13949, 13950-14014, 14015-14290, 14291-14403, 14404-14686, and 14687-14850, respectively;
wherein the method 2) comprising the steps of
a) collecting a fecal sample from the subject before and during the diet intervention or disease treatment;
b) analyzing DNA extracted from the fecal sample to determine abundance of each reference CAG selected from the group consisting of CAG ID Nos.: 1-15,
Aᵢ (abundance of CAG No.: i)=number of reads aligned to CAG No.: i/(size of CAG No.: i×number of total reads);
c) calculating ESP-index of each sample using the calculated abundance data,
ESP-Index=ln (Heip×10¹⁰×Σᵢ₌₁¹⁵Aᵢ), wherein Heip=(e^{H}−1)/14, H=−Σᵢ₌₁¹⁵ AᵢlnAᵢ, and
e) determining that the subject responds positively to the diet intervention or disease treatment if the ESP-index is increased in the sample collected during the diet intervention or disease treatment,
wherein, CAGNOs.:1-15 comprise nucleic acid sequences set forth in SEQ ID NOs.: 1-191, 192-326, 327-593, 594-835, 836-885, 886-960, 961-1097, 1098-1264, 1265-1433, 1434-1684, 1685-1833, 1834-1979, 1980-2163, 2164-2447, and 2448-2783, respectively.

2. A method for assessing the presence or the risk of development of type 2 diabetes mellitus in a subject, comprising method 3) or method 4),
wherein the method 3) comprising the steps of:
a) collecting a fecal sample from the subject;
b) analyzing DNA extracted from the fecal sample to determine abundance of each reference CAG selected from the group consisting of CAG ID Nos.: 1-64,
Aᵢ (abundance of CAG No: i)=number of reads aligned to CAG No.: i/(size of CAG No.: i×number of total reads;
c) calculating GMM-index of each sample using the calculated abundance data,
GMM-index=log (Σᵢ₌₁¹⁵Aᵢ/Σᵢ₌₁₆⁶⁴Aᵢ); and
d) determining that the subject suffers from or at a risk of developing type 2 diabetes mellitus if GMMis close to or lower than a predetermined level,
wherein, CAGNOs.:1-15 comprise nucleic acid sequences set forth in SEQ ID NOs.: 1-191, 192-326, 327-593, 594-835, 836-885, 886-960, 961-1097, 1098-1264, 1265-1433, 1434-1684, 1685-1833, 1834-1979, 1980-2163, 2164-2447, and 2448-2783, respectively, and CAG NOs.:16-64 comprise nucleic acid sequences set forth in SEQ ID NOs.: 2784-2961, 2962-3130, 3131-3525, 3526-3747, 3748-3863, 3864-4068, 4069-4212, 4213-4393, 4394-4532, 4533-4891, 4892-4979, 4980-5116, 5117-5320, 5321-5464, 5465-5781, 5782-6279, 6280-6646, 6647-6954, 6955-7178, 7179-7613, 7614-7758, 7759-8046, 8047-8491, 8492-8546, 8547-9971, 9972-10099, 10100-10392, 10393-10502, 10503-10694, 10695-10986, 10987-11089, 11090-11262, 11263-11466, 11467-11704, 11705-12034, 12035-12113, 12114-12341, 12342-12454, 12455-12664, 12665-12825, 12826-13042, 13403-13500, 13501-13726, 13727-13949, 13950-14014, 14015-14290, 14291-14403, 14404-14686, and 14687-14850, respectively;.
wherein the method 4) comprising the steps of:
a) collecting a fecal sample from the subject;
b) analyzing DNA extracted from the fecal sample to determine abundance of each reference CAG selected from the group consisting of CAG ID Nos.: 1-15,
Aᵢ (abundance of CAG No.: i)=number of reads aligned to CAG No.: i/(size of CAG No.: i×number of total reads);
c) calculating ESP-index of each sample using the calculated abundance data,
ESP-Index=ln×10¹⁰×Σᵢ₌₁¹⁵Aᵢ), wherein Heip=(e^{H}−1)/14, H=−Σᵢ₌₁¹⁵AᵢlnAᵢ and
d) determining that the subject suffers from or at a risk of developing type 2 diabetes mellitus if the ESP-index is close to or lower than a predetermined level,
wherein, CAG NOs.:1-15 comprise nucleic acid sequences set forth in SEQ ID NOs.: 1-191, 192-326, 327-593, 594-835, 836-885, 886-960, 961-1097, 1098-1264, 1265-1433, 1434-1684, 1685-1833, 1834-1979, 1980-2163, 2164-2447, and 2448-2783, respectively.

3. The method of claim 1, wherein analysis of DNA in step b) of the method 1) and comprises the steps of obtaining the DNA sequences and aligning the obtained DNA sequences with the nucleic acid sequences set forth in SEQ ID Nos.: 1-14850; and analysis of DNA in step b) of the method 2) comprises the steps of obtaining the DNA sequences and aligning the obtained DNA sequences with the nucleic acid sequences set forth in SEQ ID Nos.: 1-2783.

4. The method of claim 2, wherein analysis of DNA in step b) of the method 3) comprises the steps of obtaining the DNA sequences and aligning the obtained DNA sequences with the nucleic acid sequences set forth in SEQ ID Nos.: 1-14850; and
analysis of DNA in step b) of the method 4) comprises the steps of obtaining the DNA sequences and aligning the obtained DNA sequences with the nucleic acid sequences set forth in SEQ ID Nos.: 1-2783.

5. The method of claim 3, wherein obtaining of DNA sequences comprises the steps of obtaining raw sequence reads in the sample and processing the raw sequence reads to obtain qualified sequence reads.

6. The method of claim 4, wherein obtaining of DNA sequences comprises the steps of obtaining raw sequence reads in the sample and processing the raw sequence reads to obtain qualified sequence reads.

7. The method of claim 5, wherein the raw sequence reads are obtained by a PCR-based high-throughput sequencing technique.

8. The method of claim 5, wherein the processing of the raw sequence reads comprises removal of adapters, trimming of sequences at 3′end until reaching the first nucleotide with a quality threshold higher than 20, removal of short sequences, and removal of sequences aligned to human genome.

9. The method of claim 3, wherein the alignment of DNA sequences uses seed-and-extend strategy.

10. The method of claim 9, wherein the sequences with no mismatch in seed sequence are used to determine the abundance of each reference CAG in step b).

11. The method of claim 9, wherein the seed sequence is 4-31 bp in length, preferably, the seed is 20 bp in length.

12. The method of claim 1, wherein during the diet intervention or disease treatment, the fecal sample is collected one week, two weeks, three weeks, and/or four weeks after the diet intervention or disease treatment begins.

13. The method of claim 1, wherein in the method 1) the subject is determined to respond positively to the diet intervention or disease treatment when the GMM-index becomes close to or higher than a predetermined level during the diet intervention or disease treatment, preferably the predetermined level is −1.028883; and
in the method 2) the subject is determined to respond positively to the diet intervention or disease treatment when the ESP-index becomes close to or higher than a predetermined level during the diet intervention and disease treatment, preferably the predetermined level is 4.4.

14. The method of claim 2, wherein in the method 3) the predetermined level is approximately −1.028883; and
in the method 4) the predetermined level is approximately 4.4.

15. The method of claim 6, wherein the raw sequence reads are obtained by a PCR-based high-throughput sequencing technique.

16. The method of claim 6, wherein the processing of the raw sequence reads comprises removal of adapters, trimming of sequences at 3′end until reaching the first nucleotide with a quality threshold higher than 20, removal of short sequences, and removal of sequences aligned to human genome.

17. The method of claim 4, wherein the alignment of DNA sequences uses seed-and-extend strategy.

18. The method of claim 17, wherein the sequences with no mismatch in seed sequence are used to determine the abundance of each reference CAG in step b).

19. The method of claim 17, wherein the seed sequence is 4-31 bp in length, preferably, the seed is 20 bp in length.

20. A microbe, comprising one or more of a bacteria corresponding-CAG NO.1-15, wherein CAG NO.1-15 comprises nucleic acids set forth in SEQ ID NO.: 1-191, 192-326, 327-593, 594-835, 836-885, 886-960, 961-1097, 1098-1264, 1265-1433, 1434-1684, 1685-1833, 1834-1979, 1980-2163, 2164-2447, and 2448-2783 respectively.
